(19) United States
(12) Patent Application Publication
(10) Pub. No.: US 2026/0331271 A1
(43) Pub. Date: Oct. 22, 2026

(54) Quality Assurance Method, Quality Assurance Phantom and Computer-Readable Storage Medium

(71) Applicants: SUN YAT-SEN UNIVERSITY CANCER CENTER (THE AFFILIATED CANCER HOSPITAL OF SUN YAT-SEN UNIVERSITY,, GUANGZHOU (CN); Shanghai United Imaging Healthcare Co., Ltd, Shanghai (CN)

(72) Inventors: Wenzhao Sun, GUANGZHOU (CN); Xiaoyan Huang, GUANGZHOU (CN); Zhonghua Shi, Shanghai (CN); Zhenfeng Hou, Shanghai (CN); Can Liao, Shanghai (CN)

(21) Appl. No.: 19/459,110

(22) Filed: Jan. 26, 2026

(30) Foreign Application Priority Data
Jan. 24, 2025 (CN) 202510123037.1

(51) Int. Cl. G16H 40/40; G16H 20/40; G16H 30/20; G16H 40/63

(52) U.S. Cl. CPC G16H 40/40; G16H 20/40; G16H 30/20; G16H 40/63

(57) ABSTRACT

The disclosure provide a quality assurance method for performing quality assurance on a radiation delivery system, the method comprising: performing a first quality assurance operation using a quality assurance phantom; and performing a second quality assurance operation using the quality assurance phantom, wherein the first quality assurance operation is one of an imaging quality assurance operation and a machine quality assurance operation, and the second quality assurance operation is the other one of the imaging quality assurance operation and the machine quality assurance operation, and between the first quality assurance operation and the second quality assurance operation, the quality assurance phantom is automatically moved from a first position corresponding to the first operation to a second position corresponding to the second operation, or remains at a fixed position.

### CROSS-REFERENCE TO RELATED APPLICATIONS

The present disclosure claims priority under 35 U.S. C § 119(a) to Chinese Patent Application No. 202510123037.1 filed on Jan. 24, 2025 entitled “QUALITY ASSURANCE METHOD, QUALITY ASSURANCE PHANTOM AND COMPUTER-READABLE STORAGE MEDIUM”, the contents of which are incorporated herein by reference to their entirety.

### TECHNICAL FIELD

The present disclosure generally relates to the technical field of radiation delivery, particularly to a quality assurance method, a quality assurance phantom and a non-transitory computer-readable storage medium.

### BACKGROUND

In the field of radiation delivery, such as radiation therapy and radiation processing, the requirements for radiation delivery quality are exceptionally stringent. Consequently, quality assurance (QA) for the overall radiation delivery system or the device included in the system is of great importance. For instance, with advancements in medical technology, radiation therapy has become one of the three primary approaches in oncological treatment. As the key device in radiation therapy, the performance of medical linear accelerators (LINACs) directly influences the quality of treatment. Therefore, QA for medical LINACs is critically significant.

Traditionally, QA for medical LINACs involves numerous evaluation items concerning multiple performance aspects-including mechanical, dosimetry, and imaging properties-each requiring rigorous assessment. This traditionally necessitated manual replacement of quality assurance phantoms by users when evaluating different performance parameters, leading to relatively low QA efficiency.

### SUMMARY

Based on this, it is necessary to provide a quality assurance method, a quality assurance phantom, an apparatus, computer device, a non-transitory computer-readable storage medium, and a computer program product capable of improving the quality assurance efficiency of medical accelerators to address the aforementioned technical issues.

In a first aspect, the present disclosure provides a quality assurance method for performing quality assurance on a radiation delivery system. The method includes: performing a first quality assurance operation using a quality assurance phantom; and performing a second quality assurance operation using the quality assurance phantom. The first quality assurance operation is one of an imaging quality assurance operation and a machine quality assurance operation, and the second quality assurance operation is the other one of the imaging quality assurance operation and the machine quality assurance operation, and between the first and second quality assurance operations, the quality assurance phantom is automatically moved from a first position corresponding to the first operation to a second position corresponding to the second operation, or remains at a fixed position.

In one embodiment, the quality assurance phantom includes an imaging quality assurance module for the imaging quality assurance operation and a machine quality assurance module for the machine quality assurance operation.

In one embodiment, the quality assurance method includes: positioning the imaging quality assurance module at a quality assurance position of the radiation delivery system and performing the imaging quality assurance operation to determine an imaging quality assurance result; automatically moving the quality assurance phantom to position the machine quality assurance module at the quality assurance position and performing the machine quality assurance operation to determine a machine quality assurance result, or the method includes: positioning the machine quality assurance module at the quality assurance position of the radiation delivery system and performing the machine quality assurance operation to determine a machine quality assurance result; automatically moving the quality assurance phantom to position the imaging quality assurance module at the quality assurance position and performing the imaging quality assurance operation to determine an imaging quality assurance result.

In one embodiment, the imaging quality assurance module includes at least one sub-module, each sub-module being configured for being imaged to determine different imaging performance parameters, and the performing the imaging quality assurance operation to determine an imaging quality assurance result includes: acquiring a first image of each sub-module; and performing imaging performance analysis on the first images to determine an imaging quality assurance result including one or more imaging performance parameters.

In one embodiment, performing the machine quality assurance operation to determine a machine quality assurance result includes: adjusting the motion state of one or more moving parts in the radiation delivery system according to predetermined machine parameters, and acquiring second images of the machine quality assurance module under different motion states; analyzing the second images to determine the machine quality assurance result including one or more machine performance parameters.

In one embodiment, the quality assurance method includes: in response to a user instruction input by a user, controlling the radiation delivery system to perform the first quality assurance operation; after completion of the first quality assurance operation, controlling the support platform to move the quality assurance phantom disposed thereon to the second position, or maintaining the phantom at the first position, and automatically controlling the system to perform the second quality assurance operation; after completion of the second operation, providing feedback information to the user via a user interface.

In one embodiment, the imaging quality assurance module includes a uniform water sub-module and an air sub-module. The method further includes: acquiring a third image of the uniform water sub-module and a fourth image of air sub-module; determining correction parameters based on the third image and the fourth image. The correction parameters are used for correcting a target image, the target image being acquired by imaging a target subject using an imaging component of the radiation delivery system.

In one embodiment, the quality assurance method further includes: positioning the quality assurance phantom outside the radiation field of the radiation delivery system and performing a dosimetry quality assurance operation to determine a dosimetry quality assurance result.

In one embodiment, the method further includes: generating a quality assurance report based on one or more of an imaging quality assurance result, a machine quality assurance result, and a dosimetry quality assurance result. This includes: obtaining one or more of a historical imaging quality assurance result, a historical machine quality assurance result, and a historical dosimetry quality assurance result; and performing one or more of following steps (i) to (iii): (i) comparing the historical imaging quality assurance result and the imaging quality assurance result to determine an imaging quality assurance trend result, (ii) comparing the historical machine quality assurance result and the machine quality assurance result to determine a machine quality assurance trend result, and (iii) comparing the historical dosimetry quality assurance result and the dosimetry quality assurance to determine a dosimetry quality assurance trend result; and generating a quality assurance report based on one or more of the imaging quality assurance result, the machine quality assurance result and the dosimetry quality assurance result, and one or more of the imaging quality assurance trend result, the machine quality assurance trend result and the dosimetry quality assurance result.

In one embodiment, the method includes: acquiring a fifth image of the machine quality assurance module; determining target correction information based on the fifth image. The target correction information is used for characterizing the correction relationship between three-dimensional spatial coordinates and two-dimensional planar coordinates and is used for image reconstruction of raw imaging data of a target subject acquired by the imaging component to produce a target reconstructed image.

In one embodiment, the quality assurance method includes: positioning the quality assurance phantom at the quality assurance position of the radiation delivery system and performing the imaging quality assurance operation to determine the imaging quality assurance result; maintaining the phantom at the quality assurance position and performing the machine quality assurance operation to determine the machine quality assurance result, or the method includes: positioning the quality assurance phantom at the quality assurance position and performing the machine quality assurance operation to determine a machine quality assurance result; maintaining the phantom at the quality assurance position and performing the imaging quality assurance operation to determine an imaging quality assurance result.

In one embodiment, at least the procedure from the first quality assurance operation to the second quality assurance operation is performed automatically.

In a second aspect, the present disclosure provides a quality assurance phantom for use in quality assurance of a radiation delivery system. The phantom includes: an imaging quality assurance module used for an imaging quality assurance operation to determine an imaging quality assurance result; and a machine quality assurance module used for a machine quality assurance operation to determine a machine quality assurance result. The imaging quality assurance module is configured to be positioned at a quality assurance position in the radiation delivery system during the imaging quality assurance operation, and the machine quality assurance module is configured to be positioned at the quality assurance position during the machine quality assurance operation, or both of the imaging quality assurance module and the machine quality assurance module are configured to remain in a fixed position during the imaging quality assurance operation and the machine quality assurance operation.

In one embodiment, the imaging quality assurance module includes one or more of: a uniform water sub-module, an air sub-module, a contrast-to-noise ratio sub-module, a low-contrast sub-module, and a spatial resolution sub-module.

In one embodiment, the quality assurance phantom is automatically moved to switch between a first state where the imaging module is positioned at the quality assurance position and a second state where the machine module is positioned at the quality assurance position.

In a third aspect, the present disclosure provides a non-transitory computer-readable storage medium having a computer program stored thereon that, when executed by a processor, implements the step(s) of the quality assurance method described above.

In a fourth aspect, the present disclosure provides a quality assurance apparatus that uses a quality assurance phantom comprising an imaging quality assurance module and a machine quality assurance module to perform quality assurance on a radiation delivery system, apparatus including: a quality assurance module configured to perform a first quality assurance operation and a second quality assurance operation, the first operation is one of an imaging quality assurance operation and a machine quality assurance operation, and the second operation is the other one of the imaging quality assurance operation and the machine quality assurance operation, an between the first quality assurance operation and the second quality assurance operation, the quality assurance phantom is automatically moved from a first position corresponding to the first quality assurance operation to a second position corresponding to the second quality assurance operation, or remains at a fixed position.

In a fifth aspect, the present disclosure provides a quality assurance method using a quality assurance phantom for a radiation delivery system. The method includes: in response to a user instruction, controlling, by a processor, the radiation delivery device to perform a first quality assurance operation; after or at least partially concurrently with the first operation, performing a second quality assurance operation. The first operation is one of an imaging or machine quality assurance operation, and the second is the other. Between the operations, the phantom is automatically moved from a first position corresponding to the first operation to a second position corresponding to the second operation, or remains fixed.

In a sixth aspect, the present disclosure provides a computer device comprising a memory that stores a computer program and a processor executes the computer program to implement the step(s) of the quality assurance method described in the first aspect.

In a seventh aspect, the present disclosure provides a computer program product comprising a computer program that, when executed by a processor, implements the step(s) of the quality assurance method described in the first aspect.

Additional features will be set forth in part in the description which follows, and in part will become apparent to those skilled in art upon examination of the following and accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1A shows an exemplary flowchart illustration of a quality assurance method related to the present disclosure;

FIG. 1B shows an application environment illustration of the quality assurance method in an embodiment;

FIG. 2A shows an illustration of the layout and dimensions of a quality assurance phantom in an embodiment;

FIG. 2B shows the structural illustration of a quality assurance phantom in one embodiment;

FIG. 2C shows an illustration of the layout and dimensions of a quality assurance phantom in another embodiment;

FIG. 3A shows a flowchart illustration of a quality assurance method in an embodiment;

FIG. 3B shows a flowchart illustration of a quality assurance method in another embodiment;

FIG. 4 shows a flowchart illustration of a quality assurance method in yet another embodiment;

FIG. 5A shows an illustration of a first image of a contrast-to-noise ratio sub-module in one embodiment;

FIG. 5B shows an illustration of a first image of a uniform water sub-module in an embodiment;

FIG. 5C shows an illustration of a first image of a low-contrast submodule in one embodiment;

FIG. 5D shows an illustration of a first image of a spatial resolution submodule in an embodiment;

FIG. 6 shows a flowchart illustration of a quality assurance method in another embodiment;

FIG. 7 shows a projection illustration of a machine quality assurance module in an embodiment;

FIG. 8 shows a flowchart illustration of a quality assurance method in another embodiment;

FIG. 9 shows an illustration of dose information in one embodiment;

FIG. 10 shows a flowchart illustration of a quality assurance method in another embodiment;

FIG. 11 shows a flowchart illustration of a quality assurance method in another embodiment;

FIG. 12 shows a flowchart illustration of a quality assurance method in another embodiment;

FIG. 13 shows a functional illustration of a quality assurance phantom in an embodiment;

FIG. 14 shows a complete workflow illustration of quality assurance in an embodiment;

FIG. 15 shows a structural block diagram of a quality assurance device in an embodiment;

FIG. 16 illustrates a flowchart illustration of a quality assurance method in an embodiment;

FIG. 17 illustrates an internal structure illustration of a computer device in an embodiment.

### DETAILED DESCRIPTION

The following description is presented to enable any person skilled in art to make and use the present disclosure, and is provided in the context of a particular application and its requirements. Various modifications to the disclosed embodiments will be readily apparent to those skilled in art, and the general principles defined herein may be applied to other embodiments and applications without departing from the spirit and scope of the present disclosure. Thus, the present disclosure is not limited to the embodiments shown, but is to be accorded the widest scope consistent with the claims.

The terminology used herein is for the purpose of describing particular exemplary embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

These and other features, and characteristics of the present disclosure, as well as the methods of operation and functions of the related elements of structure and the combination of parts and economies of manufacture, may become more apparent upon consideration of the following description with reference to accompanying drawings, all of which form a part of the present disclosure. It is to be expressly understood, however, that the drawings are for the purpose of illustration and description only and are not intended to limit the scope of the present disclosure. It is understood that the drawings are not to scale.

Radiotherapy is one of the three major methods for tumor treatment at present. Medical linear accelerators are the main treatment devices for radiotherapy, and their performance directly affects the quality of radiotherapy. Therefore, the quality assurance of medical linear accelerators is particularly important. Medical linear accelerators are large in size, complex in structure, and integrate multiple high-precision technologies. Therefore, their mechanical performance, dosimetry performance, and imaging performance need to be strictly controlled. Usually, the quality assurance of the performance involves multiple items, each of which is completed independently and has many steps. Moreover, some of the items in quality assurance methods overlap partially with each other, and some items are carried out manually, which is highly dependent on the personal experience of medical physicists. As a result, the entire quality assurance process is not only time-consuming and labor-intensive, but also has a large variability with respect to the quality assurance result. Additionally, when different quality assurance operations directed to different performances are carried out, users need to manually change the quality assurance phantoms, which leads to low quality assurance efficiency.

Based on this, the present disclosure provides an innovative quality assurance phantom and a unique kind of quality assurance testing and analysis methods, which enable fully automated quality assurance process of the mechanical performance, dosimetry performance, and imaging performance of medical linear accelerators and have advantages of rapidity, automation, high precision, and high repeatability. The applicant wants to clarify that the term “performance” in the present disclosure may include at least one of the property, the efficacy and the quality, and the terms “image” and “picture” can be used interchangeably in the present disclosure.

In the quality assurance method proposed in the embodiments of the present disclosure, an integrated quality assurance phantom is utilized, and a complete quality assurance workflow is designed based on this integrated quality assurance phantom. When the quality assurance phantom is placed on a support platform (such as a couch, an object carrying table), the quality assurance tasks for the imaging performance and machine performance of the radiation delivery system can be automatically completed at one time based on this quality assurance workflow. This not only improves the quality assurance efficiency but also avoids various problems that reduce the quality of quality assurance work, such as individual experience differences, lack of quality assurance tools, and unprofessional quality assurance test methods, thereby improving the quality of quality assurance work and the consistency and accuracy of quality assurance result.

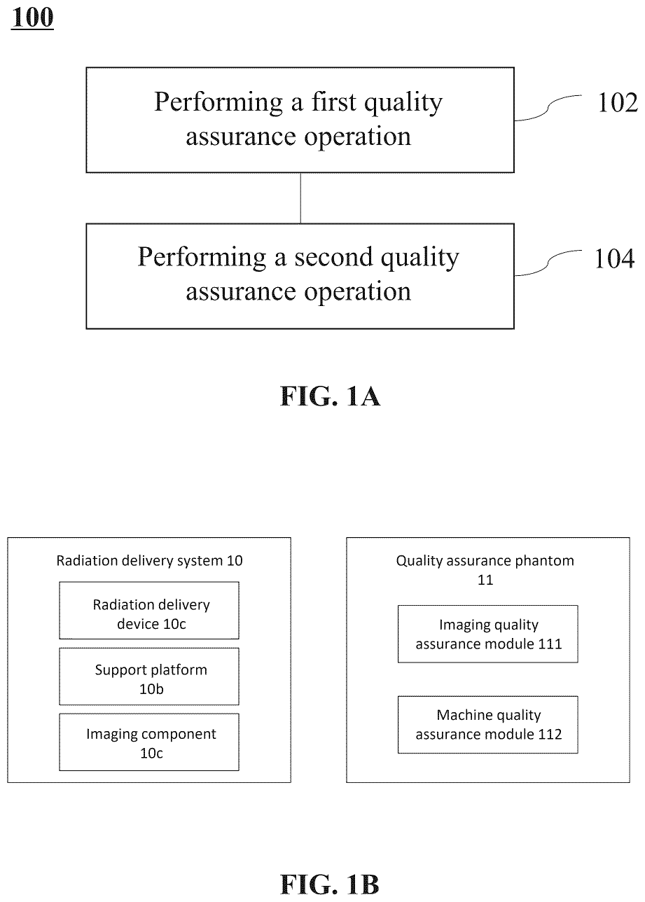

Referring to FIG. 1A, the present disclosure provides a quality assurance method 100. In the quality assurance method 100, a quality assurance phantom can be used to perform quality assurance on a radiation delivery system.

At step 102, a first quality assurance operation can be performed using a quality assurance phantom. The first quality assurance operation can be used to determine a corresponding first quality assurance result.

At step 104, a second quality assurance operation can be performed using the quality assurance phantom. The second quality assurance operation can be used to determine a corresponding second quality assurance result. The second quality assurance operation is different from the first quality assurance operation. The second quality assurance operation can be performed after the first quality assurance operation or at least partially simultaneously with the first quality assurance operation.

The first quality assurance operation can be one of an imaging quality assurance operation and a machine quality assurance operation, and the second quality assurance operation can be the other one of the imaging quality assurance operation and the machine quality assurance operation. For example, the first quality assurance operation can be an imaging quality assurance operation, and the second quality assurance operation can be a machine quality assurance operation; or, the first quality assurance operation can be a machine quality assurance operation, and the second quality assurance operation can be an imaging quality assurance operation.

Between the first quality assurance operation and the second quality assurance operation, the quality assurance phantom can be automatically moved or remain in a fixed position. For example, between the first quality assurance operation and the second quality assurance operation, the quality assurance phantom can be automatically moved from a first position corresponding to the first quality assurance operation to a second position corresponding to the second quality assurance operation, or remain in a fixed position (e.g., the first position or another position). Here, the term “move” can include various ways that cause a change in the spatial position of the entire subject (e.g., the quality assurance phantom) or a part of it, such as including but not limited to one or more of the translation, the rotation, and the tilting. In the case where the quality assurance phantom is automatically moved, for example, during the execution of the first quality assurance operation, the quality assurance phantom is positioned at the first position, and during the execution of the second quality assurance operation, the quality assurance phantom is automatically moved to a second position different from the first position. In the case where the quality assurance phantom remains in a fixed position, for example, during the execution of the first quality assurance operation and the second quality assurance operation, the quality assurance phantom remains at the first position, and the position of the quality assurance phantom remains unchanged. In some embodiments, when the quality assurance phantom is positioned at any one of the first position, the second position, and the third position, there is a position point in the quality assurance phantom that coincides with the isocenter position of the radiotherapy device. During the process from the execution of the first quality assurance operation to the execution of the second quality assurance operation, the quality assurance phantom does not need to be manually positioned. As a result, multiple quality assurance operations can be automated without the need for manual positioning of the quality assurance phantom. In some embodiments, the quality assurance phantom can be automatically moved or remain in a fixed position during the period from the completion of the first quality assurance operation to the start of the second quality assurance operation. In some embodiments, the quality assurance phantom can be automatically moved or remain in a fixed position during the period from the start of the first quality assurance operation to the completion of the second quality assurance operation.

In some embodiments, the position of at least a portion of the quality assurance phantom can be monitored during the period from the execution of the first quality assurance operation to the execution of the second quality assurance operation by a position monitoring device (e.g., a camera, a rangefinder, etc.) internal to or external to the radiation delivery system. In one example, an image of the quality assurance phantom can be captured by a camera (e.g., a depth camera or a multi-eye camera, etc.), and the position of the quality assurance phantom can be determined by a visual detection positioning algorithm (e.g., a two-dimensional positioning algorithm, a feature matching positioning algorithm, a three-dimensional visual positioning algorithm, a deep learning-driven positioning algorithm, etc.). In another example, the first relative position of the quality assurance phantom relative to the rangefinder can be measured by the rangefinder, and the relative position can be converted into absolute position of the quality assurance phantom or the second relative position of the quality assurance phantom relative to the radiation delivery device through coordinate transformation.

In a non-limiting example, the quality assurance phantom can be automatically moved or remain in a fixed position throughout the entire quality assurance method 100. In some embodiments, the quality assurance phantom can be automatically moved or remain in a fixed position during the period from the end of the first quality assurance operation to the start of the second quality assurance operation.

In another non-limiting example, before the first quality assurance operation is performed, the quality assurance phantom can be manually positioned at a quality assurance position or another position. Alternatively or additionally, after the completion of the second quality assurance operation, the position of the quality assurance phantom can be manually changed. In some embodiments, the quality assurance phantom can be manually or semi-automatically positioned at a quality assurance position (e.g., the isocenter position or a position at a predetermined distance from the isocenter position, etc.) or another position based on a laser and/or a marker, etc. For example, when the quality assurance method 100 is implemented, the imaging quality assurance module or the machine quality assurance module can be manually positioned at the quality assurance position; or, the imaging quality assurance module can be manually positioned at the quality assurance position, and then the quality assurance phantom can be automatically controlled to move such that the machine quality assurance module is located at the quality assurance position; or, the imaging quality assurance module in the quality assurance phantom can be automatically controlled to be positioned at the quality assurance position, and then the machine quality assurance module can be controlled to be positioned at the quality assurance position. Additionally, when controlling the movement of the quality assurance phantom, a movement component related to the quality assurance phantom can be set in order to enable that the movement the quality assurance phantom on the support platform (e.g., a couch, an object carrying table, etc.) can be controlled to move via the movement component without moving the support platform; or, the support platform can be controlled to move to drive the movement of the quality assurance phantom while the quality assurance phantom remains stationary relative to the support platform.

In this disclosure, automatically moving the quality assurance phantom can include controlling the movement of the quality assurance phantom on the support platform, and automatically positioning the quality assurance phantom can include positioning the quality assurance phantom at a designated position (e.g., the quality assurance position) by the way of automatically moving the quality assurance phantom. The position of the quality assurance phantom can be determined by using a camera to capture an image of the quality assurance phantom, as well as one or more of a marker on the couch and a laser projection of a laser on the couch or the quality assurance phantom, etc. Alternatively or additionally, the position of the quality assurance phantom can be determined by using a rangefinder or other devices.

In one embodiment, the first quality assurance result can be determined after the completion of the second quality assurance operation. In another embodiment, the first quality assurance result can be determined before the second quality assurance operation is performed or during the second quality assurance operation.

In some embodiments, the determination of one or more of the first quality assurance result and the second quality assurance result can be completed during the period when the quality assurance phantom is automatically moved or remains in a fixed position, or after the period when the quality assurance phantom is automatically moved or remains in a fixed position.

As an example, in the quality assurance method 100, at least the procedure which is from the start of the first quality assurance operation to the end of the second quality assurance operation can be automatically performed. For instance, Steps 102 and 104 in the quality assurance method 100 are automatically implemented.

As a non-limiting specific example, the quality assurance method 100 can be implemented as an automated quality assurance workflow based on user interaction. For example, Step 102 can specifically include: in response to a user instruction input by the user, performing the first quality assurance operation. Step 104 can specifically include: after the first quality assurance operation is completed, automatically moving the quality assurance phantom to the second position, or keeping the quality assurance phantom at the first position and performing the second quality assurance operation, and providing feedback information to the user after the second quality assurance is completed. The user can input user instructions in various ways. For instance, the user can input user instructions by using at least one of a touchscreen or button at the radiotherapy delivery device in the radiotherapy delivery system, a wired remote controller or a wireless remote controller, and a mouse, keyboard or touchscreen at a computer device communicatively connected with the radiotherapy delivery device. The feedback information provided to the user can have various types. For example, the feedback information can include at least one of a notification of the end of the quality assurance process, a display of the quality assurance result, and an alarm of possible problems in the quality assurance process. In this example, except for the operation of the user inputting the user instruction, all other operations of the quality assurance method 100 are automatically completed. That is, one-click quality assurance can be realized.

In some embodiments, the quality assurance phantom can include an imaging quality assurance module and a machine quality assurance module. Optionally, the quality assurance phantom can include other various modules such as a dose quality assurance module.

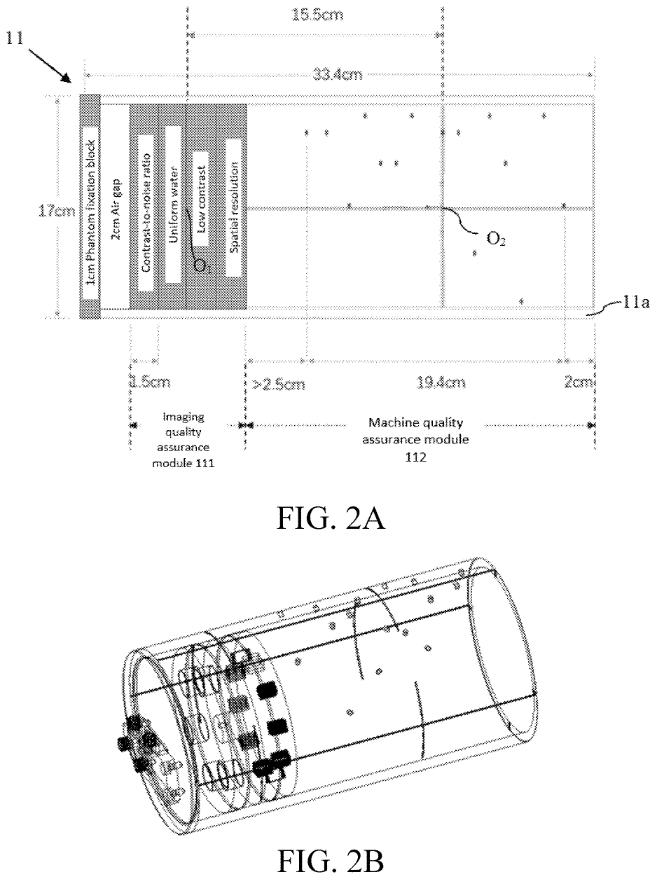

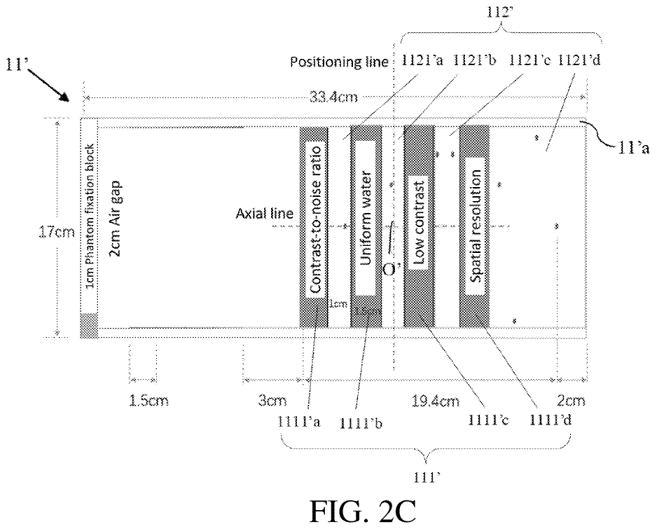

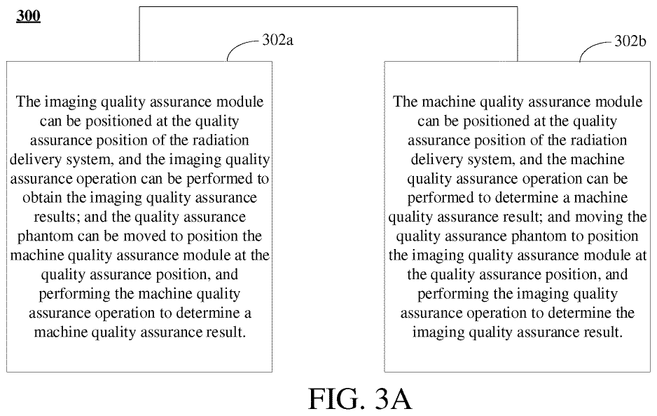

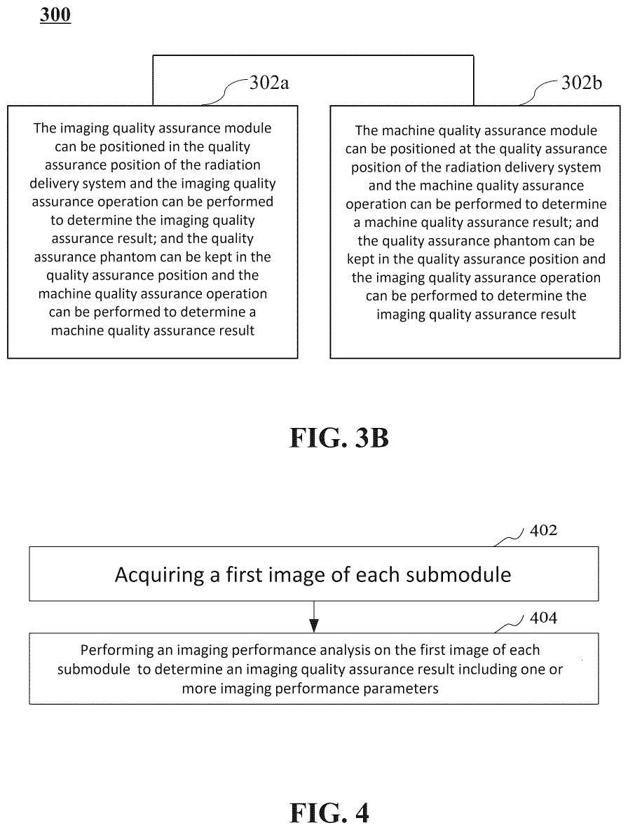

In some embodiments, in step 102, the imaging quality assurance module (or the machine quality assurance module) can be positioned at the quality assurance position of the radiotherapy delivery system (for example, the isocenter position of the radiotherapy delivery system), and the imaging quality assurance operation (or the machine quality assurance operation) can be performed to determine the imaging quality assurance result (or the machine quality assurance result); in step 104, the quality assurance phantom can be automatically moved so that the machine quality assurance module (or the imaging quality assurance module) is located at aforementioned quality assurance position, and the machine quality assurance operation (or the imaging quality assurance operation) can be performed to determine the machine quality assurance result. This embodiment will be further described in combination with the figures such as FIG. 2A, FIG. 2B, and FIG. 3A and the corresponding descriptions in the following text.

In other embodiments, in step 102, the quality assurance phantom can be positioned at the quality assurance position of the radiotherapy delivery system, and the imaging quality assurance operation (or the machine quality assurance operation) can be performed to determine the imaging quality assurance result (or the machine quality assurance result); and the quality assurance phantom can be kept at aforementioned quality assurance position, and the machine quality assurance operation (or the imaging quality assurance operation) can be performed to determine the machine quality assurance result (or the imaging quality assurance result). This embodiment will be further described in combination with the figures such as FIG. 2C and FIG. 3B and the corresponding descriptions in the following text.

The quality assurance method 100 provided by the embodiments of the present disclosure can be applied in application environment as shown in FIG. 1B. In this environment, the radiotherapy delivery system 10 can include a radiotherapy delivery device 10a and a support platform 10b (such as a couch, an object carrying table, etc.), and the radiotherapy delivery device 10a can at least include a medical accelerator, which can include but is not limited to an electron induction accelerator, a linear accelerator, or a cyclotron accelerator, etc. When quality assurance operations is performed for the radiotherapy delivery system 10, a quality assurance phantom 11 can be used to assist in performing the quality assurance operations, and the quality assurance phantom 11 can be placed on the support platform 10b, and the radiation produced by the radiotherapy delivery device 10a can be applied to the quality assurance phantom 11 to perform the corresponding quality assurance operations. Optionally, the quality assurance operations can include one or more of an imaging quality assurance operation, a machine quality assurance operation, and a dosimetry quality assurance operations. For example, the quality assurance phantom 11 may include an imaging quality assurance module 111 and a machine quality assurance module 112. When the quality assurance phantom 11 is used to perform quality assurance on the radiation delivery system 10, the imaging quality assurance module 111 and the machine quality assurance module 112 can be placed along the moving direction (e.g., the front-to-back moving direction, the left-to-right moving direction, etc.) of the support platform 10b of the radiation delivery system 10 Moreover, for the imaging quality assurance module 111 (which can be referred to as the IQA (Imaging Quality Assurance) module), it may include one or more sub-modules for assisting in the detection of different imaging performance parameters. For example, the imaging quality assurance module 111 may include, but is not limited to, at least one of a contrast-to-noise ratio sub-module, a uniform water sub-module, a low contrast sub-module, and a spatial resolution sub-module, etc., for analyzing imaging performance parameters such as CT value linearity, contrast-to-noise ratio, noise, CT value uniformity, low contrast, spatial resolution, etc.

For the machine quality assurance module 112 (which can be referred to as the MQA (Machine Quality Assurance) module), it may contain one or more metal balls, and the coordinates of each metal ball relative to the center of the quality assurance phantom are known. For example, the machine quality assurance module 112 may adopt a hollow tubular design, with the ball embedded in the tube wall, which can improve the imaging contrast of the ball and reduce the weight of the entire quality assurance phantom. The machine quality assurance module 112 can be used to check the machine performance parameters such as isocenter size, performance of the machine's moving parts, etc.

Referring to FIGS. 2A to 2C, the structural schematic illustration of exemplary quality assurance phantoms is shown. FIG. 2A is a structural schematic illustration of a quality assurance phantom 11, FIG. 2B is a three-dimensional overall structural schematic illustration of the quality assurance phantom, and FIG. 2C is a structural schematic illustration of another quality assurance phantom 11′.

As shown in FIGS. 2A and 2B, in some embodiments, the imaging quality assurance module 111 may be set on a first side of the quality assurance phantom 11 (for example, the left side of the quality assurance phantom 11 in FIG. 2A), and the machine quality assurance module 112 may be set on the second side opposite to the first side of the quality assurance phantom (for example, the right side of the quality assurance phantom 11 in FIG. 2A). Optionally, the imaging quality assurance module 111 and the machine quality assurance module 112 may be disposed adjacent to each other.

As shown in FIG. 2C, in some embodiments, at least one part of the imaging quality assurance module 111′ may be set between multiple parts of the machine quality assurance module 112′. Alternatively or additionally, at least one part of the machine quality assurance module 112′ may be set between multiple parts of the imaging quality assurance module 111′. For example, the imaging quality assurance module 111′ may include a plurality of first parts 1111′ (1111′a to 1111′d), and the machine quality assurance module 112′ may include more than one second part 1121′ (1121′a to 1121′d), with part 1111′ and part 1121′ interleaved with each other. As an example, the first part 1111′, may include a plurality of submodules described in detail later.

By way of example, the quality assurance phantoms 11, 11′ may also include a phantom fixation block, which may be used to locate or replace modules of each phantom. The quality assurance phantom 11, 11′ can also include an air gap, through which the fixed block of the phantom and the imaging quality assurance module 111, 111′ can be isolated from each other. It should be noted that the size of the quality assurance phantom, the size of each module, arrangement order and the number of metal balls shown in FIGS. 2A to 2C can be adjusted according to actual situation of the machine (e.g., the radiation delivery device 10a), and are not limited to the aforementioned examples.

In an exemplary embodiment, as shown in FIG. 3A, a quality assurance method 300 is provided. The quality assurance method 300 can be used for quality assurance of a radiation delivery device using quality assurance phantoms, such as the quality assurance module 11 shown in FIGS. 2A and 2B. The application of the method to the radiation delivery system in FIG. 1B is illustrated as an example. The method is either applied to a radiation delivery in the radiation delivery system, or a processor or controller corresponding to the radiation delivery system, which may be either independent of the radiation delivery device or integrated into the radiation delivery device, without limitation in this embodiment. Based on this, the quality assurance method 300 may include the following Steps 302a or 302b.

In Step 302a, the imaging quality assurance module can be positioned at the quality assurance position of the radiation delivery system, and the imaging quality assurance operation can be performed to obtain the imaging quality assurance result; and the quality assurance phantom can be moved to position the machine quality assurance module at the quality assurance position, and performing the machine quality assurance operation to determine a machine quality assurance result.

The quality assurance position of the radiation delivery system may be an isocenter position of the radiation delivery system, or be in a predetermined range relative to the isocenter position. The isocenter can be a virtual point in the radiation delivery system. For example, the isocenter can be indicated by a plurality of lasers (e.g., at least three lasers) on the walls of the radiation room as a radiation target point. In the process of the radiation delivery, the isocenter of the radiation delivery system can be used as a reference center of the simulation positioning, a treatment center determined in the radiation delivery plan generation step, etc. The isocenter of the radiation delivery system can also be used in the positioning in the treatment implementation step, the plan verification step, the quality assurance step, etc.

For example, when the quality assurance operation is performed for a radiation delivery system, an imaging quality assurance operation may be performed, followed by a machine quality assurance operation. In the process of imaging quality assurance, the imaging quality assurance module in the quality assurance phantom needs to be moved to the quality assurance position of the radiation delivery system. For example, the position point (e.g., center of mass) in the imaging quality assurance module can be aligned with the intersection point of the lights emitted from a plurality of lasers of the radiation delivery system. The imaging quality assurance operation can be performed based on the predetermined imaging quality assurance workflow and the imaging quality assurance result can be obtained.

It should be noted that the imaging quality assurance module or machine quality assurance module can be manually positioned in the quality assurance position during the quality assurance process. Alternatively, the imaging quality assurance module can be manually positioned at the quality assurance position, and then the machine quality assurance phantom can be controlled to position the machine quality assurance module at the quality assurance position. Alternatively, the imaging quality assurance module in the quality assurance phantom can be automatically controlled to locate at the quality assurance position, and then the machine quality assurance module can be controlled to locate at the quality assurance position. Thereby the manual movement, or semi-automatic movement, or fully automatic movement of the quality assurance phantom can be implemented. In addition, when the quality assurance phantom is controlled to move, the moving parts related to the quality assurance phantom can be set so that the movement of the quality assurance phantom on the support platform (such as a couch, an object carrying table, etc.) can be controlled by the moving parts while the support platform stays stationary. In other embodiments, the support platform can be controlled to drive the movement of the quality assurance phantom while the quality assurance phantom stays stationary relative to the support platform.

By way of example, when the support platform is controlled to driving the movement of the quality assurance phantom, the quality assurance phantom can be placed at a predetermined position of the support platform, and the support platform can be controlled to move (for example, forward and backward translation and/or up and down translation, tilt or rotation around the length direction, width direction or height direction, etc.), so that the support platform drives the movement of the quality assurance phantom, and in turn the imaging quality assurance module in the quality assurance phantom can be placed at the quality assurance position of the radiation delivery system. During this period, the quality assurance phantom can be in a static state relative to the support platform. Furthermore, when the support platform is controlled to move, the support platform can be controlled to start moving or stop moving under auxiliary observation of the user, or the support platform can be controlled by automatic control algorithm to move until the imaging quality assurance module in the quality assurance phantom on the support platform is located at the quality assurance position of the radiation delivery system. Moreover, in addition to driving the quality assurance phantom by controlling the support platform, the quality assurance phantom can also be moved by manual or robot, or a moving component for moving the quality assurance phantom can be set on the support platform, and the quality assurance phantom can be moved by the moving component when the support platform stays still.

That is, the above quality assurance method can optionally be an automatic quality assurance method which can be applied to the processor. When the quality assurance phantom is operated, the support platform can be controlled by the processor to move so as to locate the imaging quality assurance module on the support platform at the quality assurance position of the radiation delivery system, and the imaging quality assurance operation can be performed to determine the imaging quality assurance result; and the support platform can be controlled by the processor to move so as to locate the machine quality assurance module on the support platform at the quality assurance position, and the machine quality assurance operation is performed to determine a machine quality assurance result. Alternatively or additionally, the aforementioned automatic quality assurance method can be applied to the processor and when the quality assurance phantom is operated, the support platform is controlled by the processor to move to position the machine quality assurance module on the support platform at the quality assurance position of the radiation delivery system, and the machine quality assurance operation is performed to obtain the machine quality assurance result; and the support platform is controlled by the processor to move so as to locate the imaging quality assurance module on the support platform at the quality assurance position, and the imaging quality assurance operation is performed to determine the imaging quality assurance result. The automatic quality assurance method can realize automatic quality assurance process. That is, when the user places the quality assurance phantom integrated with multiple different functional modules on the support platform, the movement of the quality assurance phantom can be realized by automatic control means, and the quality assurance operations of different dimensions (including but not limited to imaging quality assurance, machine quality assurance and dose quality assurance) of the radiation delivery system can be automatically realized through different functional modules in the quality assurance phantom. During the quality assurance process, there is no need for the user to participate in the quality assurance process, nor for the user to frequently move or replace the quality assurance module manually, which can not only save manpower, but also avoid the situation of quality assurance error caused by inaccurate placement of quality assurance module in the process of human operation. Because automatic control can more accurately realize the control of placing the different modules of the quality assurance module at the quality assurance position, it can improve accuracy of quality assurance operation, reduce quality assurance errors, and improve the overall quality assurance efficiency.

Furthermore, when the imaging quality assurance module is located at the quality assurance position of the radiation delivery system, the imaging quality assurance operation can be performed based on the predetermined imaging quality assurance workflow to obtain the imaging quality assurance result. By way of example, the radiation delivery system can be controlled to scan the imaging quality assurance module to obtain the first image, and then the imaging quality assurance result can be obtained by analyzing the first image. The imaging quality assurance result can include at least one of the imaging performance parameters such as contrast, uniformity, resolution and noise of the first image.

By way of example, when the image scan is performed, a first image of a different modalities (such as Cone Beam Computer Tomography (CBCT) image, which is also known as Cone Beam CT image, as well as Fan Beam Computer Tomography (FBCT) image, which is also known as Fan Beam CT image) can be acquired by scanning. Based on the first image of different modes, the imaging quality assurance operations of different modalities can be realized, such as CBCT quality assurance, FBCT quality assurance, etc.

After the imaging quality assurance operation is completed, the support platform can be controlled to drive the quality assurance phantom to move so as to locate the machine quality assurance module at the quality assurance position of the radiation delivery system, and the machine quality assurance operation can be performed to obtain a machine quality assurance result.

According to the operation process of imaging quality assurance mentioned above, the machine performance quality assurance of the radiation delivery system can be realized. For example, based on the integrated quality assurance phantom, the support platform can be controlled to drive the quality assurance phantom to move, so that the machine quality assurance module is located in the quality assurance position of the radiation delivery system. In this case, the machine quality assurance operation is performed based on the predetermined machine quality assurance workflow to determine a machine quality assurance result. The movement control mode of the quality assurance phantom can refer to above description of the relevant content of imaging quality assurance, which will not be repeated here.

For example, when the machine quality assurance module is located at the quality assurance position of the radiation delivery system, the machine parameters of the radiation delivery system can be adjusted, and the second image of the machine quality assurance module under different machine parameters can be acquired. Then, a machine quality assurance result can be obtained by analyzing the second image under different machine parameters. The machine quality assurance result can include at least one of the machine performance parameters such as isocenter accuracy, motion accuracy of different moving parts, and motion accuracy of different beam limiting parts.

At Step 302b, the machine quality assurance module can be positioned at the quality assurance position of the radiation delivery system, and the machine quality assurance operation can be performed to determine a machine quality assurance result; and the quality assurance phantom can be moved to position the imaging quality assurance module at the quality assurance position, and the imaging quality assurance operation can be performed to determine the imaging quality assurance result.

That is, during the quality assurance operation of the radiation delivery system, the machine quality assurance operation may be performed, followed by the imaging quality assurance operation. That is, the machine quality assurance module is positioned at the quality assurance position of the radiation delivery system to perform the machine quality assurance operation, and then the imaging quality assurance module is moved to the quality assurance position to perform the imaging quality assurance operation. The details of machine quality assurance operation and imaging quality assurance operation can refer to the relevant contents described in combination with Step 302a above and will not be repeated here.

For example, when the above quality assurance method is an automatic quality assurance method implemented by a processor, the support platform can be controlled by the processor to position the machine quality assurance module on the support platform at the quality assurance position of the radiation delivery system, and the machine quality assurance operation can be performed to determine a machine quality assurance result; and the support platform can be controlled by the processor to move so as to position the imaging quality assurance module on the support platform at the quality assurance position, and the imaging quality assurance operation can be performed to determine the imaging quality assurance result.

Furthermore, in the case where the imaging quality assurance result and the machine quality assurance result is obtained, a quality assurance report can be generated based on one or more of the imaging quality assurance result and the machine quality assurance result. Alternatively, based on one or more of the imaging quality assurance result and the machine quality assurance result, as well as the corresponding one or more of the imaging quality assurance trend result and the machine quality assurance trend result, the quality assurance reports can be generated. The imaging quality assurance trend result can be obtained after comparison analysis of the historical imaging quality assurance result and the imaging quality assurance result, and the machine quality assurance trend result can be obtained after comparison analysis of the historical machine quality assurance result and the machine quality assurance result.

The above quality assurance method can be applied to the radiation delivery system, and the quality assurance phantom including the imaging quality assurance module and the machine quality assurance module can be used to perform the quality assurance of the radiation delivery system. The quality assurance method includes: positioning the imaging quality assurance module at the quality assurance position of the radiation delivery system and performing the imaging quality assurance operation to determine the imaging quality assurance result, and moving the quality assurance phantom to position the machine quality assurance module at the quality assurance position and performing the machine quality assurance operation to determine a machine quality assurance result; or, positioning the machine quality assurance module at the quality assurance position of the radiation delivery system and performing the machine quality assurance operation to determine a machine quality assurance result, and moving the quality assurance phantom to position the imaging quality assurance module at the quality assurance position and performing the imaging quality assurance operation to determine the imaging quality assurance result.

In an exemplary embodiment, as shown in FIG. 3B, an alternative quality assurance method 300′ is provided. The quality assurance method 300′ can be used for quality assurance of the radiation delivery system using a quality assurance phantom as shown in FIG. 2C. This approach can be applied to the radiation delivery system in FIG. 1B. The quality assurance method 300′ may include the following Step 302′a or Step 302′b.

At Step 302′a, the imaging quality assurance module can be positioned at the quality assurance position of the radiation delivery system and the imaging quality assurance operation can be performed to determine the imaging quality assurance result; and the quality assurance phantom can be kept in the quality assurance position and the machine quality assurance operation can be performed to determine a machine quality assurance result.

At Step 302′b, the machine quality assurance module can be positioned at the quality assurance position of the radiation delivery system and the machine quality assurance operation can be performed to determine a machine quality assurance result; and the quality assurance phantom can be kept in the quality assurance position and the imaging quality assurance operation can be performed to determine the imaging quality assurance result.

Therefore, according to the quality assurance method 300′, the imaging quality assurance operation and the machine quality assurance operation can be performed in situ without moving the quality assurance phantom, so that the error of the quality assurance result caused by moving the quality assurance phantom can be avoided.

As a non-limiting example, the imaging component 10c in the radiation delivery system 10 can be controlled to perform imaging with respect to the imaging quality assurance module and/or the machine quality assurance operation when an imaging quality assurance operation and/or machine quality assurance operation is performed. The imaging component 10c can be included in, and/or independent of, the radiation delivery device 10a. For example, when an imaging quality assurance operation is performed, the imaging component 10c can be rotated one or more turns to image the imaging quality assurance module to acquire data for imaging quality assurance. Alternatively or additionally, during the machine quality assurance operation, imaging may be performed on the machine quality assurance module when the gantry of the radiation delivery device is moved to one or more predetermined positions to acquire data for machine quality assurance. As an example, the Imaging component 10c can include an Electronic Portal Imaging Device (EPID), Cone Beam CT (CBCT), Fan Beam CT (FBCT), and the like. In some embodiments, different imaging components can be used for different quality assurance operations. For example, CBCT can be used for imaging when the imaging quality assurance operation is performed, and EPID can be used for imaging when the machine quality assurance operation is performed.

In the quality assurance method according to the embodiment of this disclosure, an integrated quality assurance phantom is provided, and a quality assurance workflow is designed based on the integrated quality assurance phantom. When the quality assurance phantom is placed on a support platform (such as a couch, an object carrying table, etc.), based on the quality assurance workflow, the quality assurance tasks of imaging performance and machine performance of the radiation delivery system can be automatically realized at one time. It can not only improve the efficiency of quality assurance, but also avoid various problems that reduce the quality of quality assurance work such as individual experience differences, lack of quality assurance tools, and unprofessional quality assurance test methods, so as to improve the quality of quality assurance work and improve the consistency and accuracy of quality assurance result.

In an exemplary embodiment, the imaging quality assurance module can include at least one submodule, each of which is used to be imaged to determine different imaging performance parameters. Referring to the above FIG. 2A to FIG. 2C, each layer of the imaging quality assurance module can correspond to a sub-module. For example, if the sub-module is a contrast-to-noise ratio sub-module, it can be used for the quality detection of contrast-to-noise ratio of the image, and the corresponding imaging performance parameter is contrast-to-noise ratio. On the basis of above examples, as shown in FIG. 4, the imaging quality assurance operations being performed to determine the imaging quality assurance result as illustrated in Steps 302a (or 302′a) and 302b (or 302′b) above can include the following Steps 402 to 404.

At Step 402, the first image of each submodule can be acquired.

By way of example, the radiation delivery system can scan each sub-module of the imaging quality assurance module respectively to obtain the first image corresponding to each sub-module. The first image is the sectional image corresponding to the submodule.

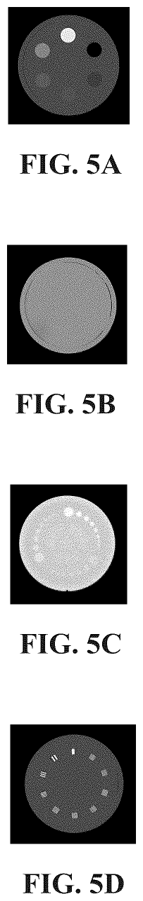

By way of example, a radiation delivery system can perform tomography of an imaging quality assurance module to obtain the first image corresponding to each sub-module. For example, referring to the above FIG. 2A to 2C, the radiation delivery system can scan the contrast-to-noise ratio sub-module to obtain the first image corresponding to the contrast-to-noise ratio sub-module, as shown in FIG. 5A; the radiation delivery system can scan the uniform water sub-module to obtain the corresponding first image of the uniform water sub-module, as shown in FIG. 5B; the radiation delivery system can scan the low-contrast submodule to obtain the corresponding first image of the low-contrast submodule, as shown in FIG. 5C; and the radiation delivery system can scan the spatial resolution sub-module to obtain the corresponding first image of the spatial resolution sub-module, as shown in FIG. 5D.

At Step 404, the imaging performance analysis can be performed on the first image of each sub-module to determine the imaging quality assurance result.

The imaging quality assurance result can include one or more imaging performance parameters (also known as imaging quality parameters). For example, the radiation delivery system can perform the imaging performance analysis of the first image of each sub-module separately to obtain the corresponding imaging performance parameters for each sub-module. Then, the imaging quality assurance result can be determined based on the imaging performance parameters of each sub-module. It should be noted that after the imaging performance analysis is performed on the first image of a submodule, at least one imaging performance parameter corresponding to the submodule can be obtained. For example, after the first image corresponding to the contrast-to-noise ratio sub-module is analyzed, the contrast-to-noise ratio parameter can be obtained; after the first image corresponding to the uniform water sub-module is analyzed, the CT value uniformity parameter can be obtained; after the first image corresponding to the low-contrast sub-module is analyzed, the low-contrast parameter can be obtained; after the first image corresponding to the spatial resolution submodule is analyzed, the spatial resolution parameter can be obtained.

For example, the radiation delivery system can also combine the first images of multiple sub-modules for comprehensive analysis to obtain the corresponding imaging performance parameters. For example, through the comprehensive analysis of the first image of multiple sub-modules, imaging performance parameters such as CT value accuracy, CT value linearity, noise, and reconstruction geometric accuracy can also be determined.

Based on this, after the imaging performance analysis is performed on the first image of each sub-module, the imaging quality assurance result including one or more imaging performance parameters can be obtained.

In this embodiment, the imaging quality assurance module can include at least one submodule for being imaged to determine different imaging performance parameters. Based on this, the delivery system can acquire the first image of each sub-module, and perform the imaging performance analysis on the first image of each sub-module to obtain the imaging quality assurance result including one or more imaging performance parameters. Using this method, the quality assurance of imaging performance can be realized through the imaging quality assurance module in the integrated quality assurance phantom, and the efficiency of quality assurance of imaging performance can be improved.

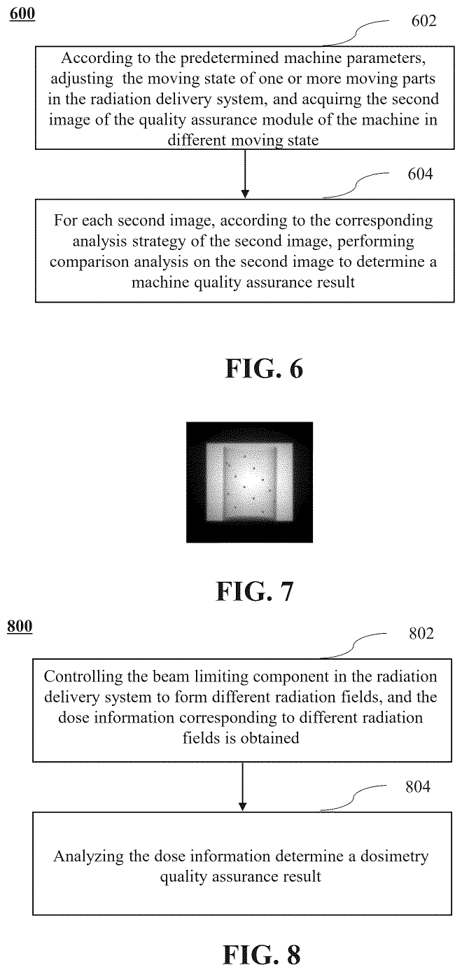

In an exemplary embodiment, as shown in FIG. 6, the machine quality assurance operations are performed in Steps 302a (or 302′a) and 302b (or 302′b) above, and the step of determining a machine quality assurance result can include Steps 602 to 604.

At Step 602, according to the predetermined machine parameters, the moving state of one or more moving parts in the radiation delivery system can be adjusted, and the second image of the quality assurance module of the machine in different moving states can be acquired.

The moving parts in the radiation delivery system may include a supporting platform (such as a couch, an object carrying table, etc.), a gantry, a flat panel detector (such as an EPID or other type of flat panel detector), a multi-leaf collimator, a jaw, a primary collimator, etc. The predetermined machine parameters can include the motion parameters (such as speed, position, etc.), state parameters (such as signal link integrity, temperature, material information and the like of the related control circuits of each part) of one or more of moving parts.

As an example, for each moving part, the motion state of the moving part in a radiation delivery system can be adjusted according to the machine parameters corresponding to that moving part. The motion state can include the state of the moving part under different motion parameters, for example, different positions of the support platform. By adjusting the position state of the support platform, the motion accuracy of the support platform can be checked.

For example, during the quality assurance of the machine performance of the radiation delivery system, the motion state of the moving part can be adjusted according to the different motion parameters of each moving part, and the second image of the machine quality assurance module under different motion states of each moving part can be obtained, as shown in FIG. 7. For example, the second image of the machine quality assurance module under different states of the support platform can be obtained, the second image of the machine quality assurance module under different states of the gantry can be obtained, etc. It should be stated that when the state of one moving part is adjusted, the state of the other moving parts can be kept unchanged.

At Step 604, for each second image, according to the corresponding analysis strategy of the second image, the comparison analysis can be performed on the second image to determine a machine quality assurance result.

The machine quality assurance result may include one or more machine performance parameters (also known as machine quality parameters). The machine performance parameters may include, but are not limited to, isocenter accuracy, support platform motion accuracy, gantry motion accuracy, flat panel detector motion accuracy, multi-leaf collimator (MLC) motion accuracy, jaw motion accuracy, primary collimator motion accuracy, Image-Guided Radiation Therapy (IGRT) accuracy, flat panel detector dark field monitoring, zero position repeatability of support platform, etc. The machine state parameters may include, but are not limited to, failure status (e.g., whether one or more of the support platform, gantry, flat panel detector, multi-leaf collimator, jaw is faulty or cannot work), power supply connection status, etc.

For each moving part, in the case of acquiring the second image under different motion states corresponding to the moving part, the second image under different motion states corresponding to the moving part can be analyzed to obtain the motion accuracy of the moving part. For example, the motion of the support platform can be controlled according to the parameters of the support platform, and multiple second images during the movement of the support platform can be acquired. The motion accuracy of the support platform can be determined by performing comparison analysis on each second image during the movement of the support platform. Optionally, for the second images acquired under different motion states, the corresponding analysis strategy for each second image can be set respectively, and for each second image, according to the corresponding analysis strategy of the second image, the second image can be analyzed to obtain the corresponding machine performance parameters of the second image. The analysis strategy corresponding to the second image can include determining that the difference between the motion information determined based on the second image and the corresponding predetermined motion information of the second image is less than the predetermined difference threshold.

By way of example, the machine performance quality assurance may also include performance testing of beam limiting components in a radiation delivery system, and accordingly, machine performance parameters may also include motion accuracy of beam limiting components. On this basis, the quality assurance operation of the machine can be carried out to determine the quality assurance result of the machine. For example, when the quality assurance phantom is positioned in the radiation field of the radiation delivery system, the beam limiting components in the radiation delivery system can be controlled to form different radiation fields, and the motion accuracy of the beam limiting components can be determined according to the measured radiation fields. Optionally, when the radiation field of the beam limiting component is measured, the field image of the beam limiting component can also be acquired, so that the size and position of the radiation field can be measured based on the field image, and the motion accuracy of the beam limiting component can be determined according to the measured size and position of the radiation field.

Optionally, the beam limiting components may include, but are not limited to, one or more of the MLC, jaw, and primary collimator. By using different beam limiting components to form different radiation fields, the motion accuracy of different components (e.g., MLC, jaw, flat panel detector, etc.) can be determined.

In this embodiment, when the machine performance quality assurance is performed, the moving state of one or more moving parts in the radiation delivery system can be adjusted according to predetermined machine parameters, and the second image of the machine quality assurance module under different moving states can be acquired. Then, for each second image, according to the corresponding analysis strategy of the second image, the second image is analyzed, and a machine quality assurance result including one or more machine performance parameters are obtained. Using this method, the quality assurance detection of machine performance can be realized through the machine quality assurance module in the integrated quality assurance phantom, and the quality assurance detection efficiency of machine performance can be improved.

In an exemplary embodiment, a quality assurance operation for a radiation delivery system may include a dosimetry quality assurance operation for a radiation delivery system in addition to an imaging quality assurance operation and a machine quality assurance operation. On the basis of above examples, above quality assurance method may also include positioning the quality assurance phantom outside the radiation field of the radiation delivery system and performing a dosimetry quality assurance operation to determine the dosimetry quality assurance result. As an example, a dosimetry quality assurance operation may be performed before, between, or after the first quality assurance operation.

For example, when dosimetry quality assurance is performed, assistance of the quality assurance phantom is not needed, and the quality assurance phantom can be moved outside the radiation field of the radiation delivery system. In this case, the dosimetry quality assurance operation can be performed based on the predetermined dosimetry quality assurance workflow to obtain a dosimetry quality assurance result.

For example, when the quality assurance phantom is moved out of the radiation field of the radiation delivery system, the predetermined radiation delivery plan can be performed through the radiation delivery system, and the dose information corresponding to the predetermined radiation delivery plan can be acquired, and a dosimetry quality assurance result can be obtained by analyzing the dose information. The dosimetry quality assurance result can include at least one dosimetry performance parameter, such as dose output change, beam energy change and planned gamma pass rate. Optionally, the dose information corresponding to the predetermined radiation delivery plan may include dose images corresponding to the predetermined radiation delivery plan, dose values corresponding to the predetermined radiation delivery plan, etc. For example, it may include dose images, dose values acquired during the execution of the radiation delivery according to the predetermined radiation delivery plan, etc. As an example, a predetermined radiation delivery plan may include, without limitation, plans generated for subsequent practical applications of radiation delivery, test plans for validating the performance of radiation delivery device, and the like.

It should be noted that above imaging quality assurance, machine quality assurance and dosimetry quality assurance do not make specific restrictions on the execution sequence of each performance quality assurance. The radiation delivery system can perform above imaging quality assurance, machine quality assurance and dosimetry quality assurance operations according to the sequence set by the user. The above imaging quality assurance operations, machine quality assurance operations and dosimetry quality assurance operations can also be performed in a predetermined sequence. For example, the imaging quality assurance operation can be performed, then the machine quality assurance operation can be performed, and then the dosimetry quality assurance operation can be performed. It is also possible to perform the machine quality assurance operation, then the imaging quality assurance operation, and then the dosimetry quality assurance operation. It is also possible to perform the dosimetry quality assurance operation, then the imaging quality assurance operation, and then the machine quality assurance operation. Alternatively, the dosimetry quality assurance operation can be performed, then the machine quality assurance operation, and then the imaging quality assurance operation.

Alternatively, the imaging quality assurance operation can be performed, then the dosimetry quality assurance operation, and then the machine quality assurance operation. Alternatively, the machine quality assurance operation can be performed, then the dosimetry quality assurance operation, and then the imaging quality assurance operation. That is, the dose quality assurance operation is implemented between the imaging quality assurance operation and the machine quality assurance operation. For example, the imaging quality assurance module can be positioned at the quality assurance position of the radiation delivery system, and the imaging quality assurance operation can be performed to obtain the imaging quality assurance result. Then, the quality assurance phantom can be controlled to move out of the radiation field of the radiation delivery system, and the dosimetry quality assurance operation can be performed to determine a dosimetry quality assurance result. Then, the quality assurance module can be controlled to move so that the machine quality assurance module can be located in the quality assurance position, and the machine quality assurance operation can be performed to determine a machine quality assurance result. Alternatively, the machine quality assurance module can be positioned at the quality assurance position of the radiation delivery system, and the machine quality assurance operation can be performed to obtain a machine quality assurance result. Then, the quality assurance phantom can be controlled to move out of the radiation field of the radiation delivery system, and the dosimetry quality assurance operation can be performed to determine a dosimetry quality assurance result. Then, the quality assurance phantom can be controlled to position the imaging quality assurance module at the quality assurance position, and the imaging quality assurance operation can be performed to determine the imaging quality assurance result.

In an exemplary embodiment, as shown in FIG. 8, the above process of performing dosimetry quality assurance operation to determine the dosimetry quality assurance result may include Steps 802 to 804.

At Step 802, the beam limiting component in the radiation delivery system is controlled to form different radiation fields, and the dose information corresponding to different radiation fields is obtained.

The beam limiting component in the radiation delivery system can include multi-leaf collimators (MLC), jaw, and primary collimators.

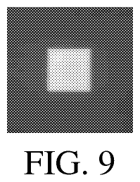

By way of example, when different radiation fields are formed by controlling the beam limiting components in the radiation delivery system, different radiation fields can be formed by adjusting one beam limiting component, or different radiation fields can be formed by adjusting multiple beam limiting components. On this basis, different dose information can be obtained under different radiation fields as shown in FIG. 9.

At Step 804, the dose information can be analyzed to determine a dosimetry quality assurance result.

For example, when the dose information corresponding to different radiation fields is obtained, a dosimetry quality assurance result can be obtained by analyzing the dose information of each field. A dosimetry quality assurance result may include one or more dosimetry performance parameters (also known as dosimetry quality parameters). The dosimetry performance parameters can include dose output and energy change parameters, plan gamma pass rate parameters, moving part tracking error parameters, etc. The dosimetry performance analysis process is not specifically limited in the embodiments of the present disclosure.

In this embodiment, during the quality assurance of dosimetry performance, different radiation fields can be formed by controlling the beam limiting component in the radiation delivery system, and the corresponding dose information of different radiation fields can be obtained. Then, each dose information can be analyzed to obtain a dosimetry quality assurance result including one or more dosimetry performance parameters. Using this method, the quality assurance test of dosimetry performance can be realized, so that the quality assurance operation of imaging performance, machine performance and dosimetry performance can be realized through a complete quality assurance workflow, and the quality assurance efficiency can be improved.

In an exemplary embodiment, a quality assurance report may also be generated based on one or more of the imaging quality assurance result, the machine quality assurance result, and the dosimetry quality assurance result, after the imaging quality assurance result, the machine quality assurance result, and the dosimetry quality assurance result have been obtained.

For example, after the imaging quality assurance, machine quality assurance, and dosimetry quality assurance are completed, the radiation delivery system can automatically generate quality assurance reports based on one or more of the imaging quality assurance result, a machine quality assurance result, and a dosimetry quality assurance result. For example, when the quality assurance report is generated, the radiation delivery system can also analyze one or more of the imaging quality assurance result, a machine quality assurance result and a dosimetry quality assurance result to determine whether the quality assurance parameter in each quality assurance result meet the corresponding quality assurance requirement. If not, the quality assurance parameter that does not meet the corresponding quality assurance conditions will be determined, and further quality assurance suggestions can be generated based on the quality assurance parameter that does not meet the corresponding quality assurance condition, and the suggestions can be added to the quality assurance report to instruct the user to re-conduct quality assurance operations on the quality assurance parameter that does not meet the corresponding quality assurance condition.

For example, when a quality assurance report is determined to be generated, the radiation delivery system can generate the quality assurance report by adding each result information to a preset report template.

By way of example, in the case that a quality assurance report is generated, the quality assurance report can also be sent to a user terminal so that the user can view the quality assurance report. The user terminal may include, but is not limited to, the user's mobile phone, laptop computer, tablet computer, the display screen of the radiation delivery system, and the like.

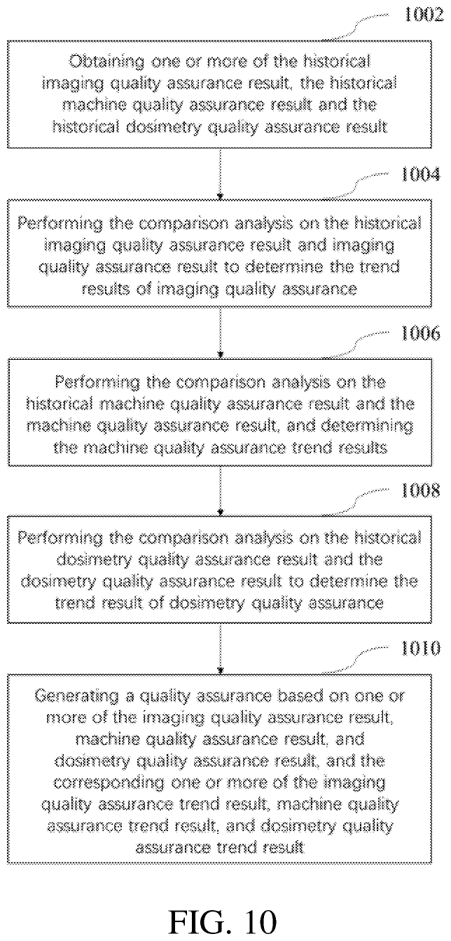

In an exemplary embodiment, the radiation delivery system may also utilize a historical imaging quality assurance result, a historical machine quality assurance result, and a historical dosimetry quality assurance result to perform comparison analysis with respect to the imaging quality assurance result, the machine quality assurance result, and the dosimetry quality assurance result, thereby generating quality assurance reports based on the comparison analysis result. Based on this, as shown in FIG. 10, the above process of generating quality assurance report based on one or more of the imaging quality assurance result, a machine quality assurance result and a dosimetry quality assurance result can include Steps 1002 to 1010.

At Step 1002, one or more of the historical imaging quality assurance result, the historical machine quality assurance result and the historical dosimetry quality assurance result can be obtained.

The historical imaging quality assurance result can include the imaging quality assurance result obtained based on at least one imaging quality assurance performed on the radiation delivery system before the current quality assurance operation. The historical machine quality assurance result can include a machine quality assurance result obtained from at least one machine quality assurance performed on the radiation delivery system before the current quality assurance operation. The historical dosimetry quality assurance result can include a dosimetry quality assurance result obtained from at least one dosimetry quality assurance performed on the radiation delivery system before the current quality assurance operation.

It should be noted that at least one historical imaging quality assurance, at least one historical machine quality assurance and at least one historical dosimetry quality assurance can include imaging quality assurance, machine quality assurance and dosimetry quality assurance obtained in the same historical quality assurance, and alternatively or additionally, can include imaging quality assurance, machine quality assurance and dosimetry quality assurance obtained in different historical quality assurances.

By way of example, the radiation delivery system may obtain historical quality assurance result from the local storage of the radiation delivery system or from other storage devices. In the embodiments of this disclosure, there is no specific limitation on the way to obtain the historical quality assurance result.

The radiation delivery system may then perform one or more of the following Steps 1004 to 1008.

At Step 1004, the comparison analysis can be performed on the historical imaging quality assurance result and imaging quality assurance result to determine the trend result of imaging quality assurance.

At Step 1006, the comparison analysis can be performed on the historical machine quality assurance result and a machine quality assurance result, and determine the machine quality assurance trend result.

At Step 1008, the comparison analysis can be performed on the historical dosimetry quality assurance result and a dosimetry quality assurance result to determine the trend result of dosimetry quality assurance.

At Step 1010, a quality assurance report can be generated based on one or more of the imaging quality assurance result, machine quality assurance result, and dosimetry quality assurance result, and the corresponding one or more of the imaging quality assurance trend result, machine quality assurance trend result, and dosimetry quality assurance trend result.

In other words, in an embodiment, the quality assurance report corresponding to the current quality assurance operation can be generated only according to the quality assurance result of the current quality assurance operation, that is, one or more of the imaging quality assurance result, the machine quality assurance result, and the dosimetry quality assurance result of the current quality assurance operation. In another embodiment, the quality assurance result of the current secondary quality assurance operation can also be compared with the historical quality assurance result of at least one previous quality assurance operation to obtain the change trend of each quality assurance parameter, and in turn according to the quality assurance result of the current secondary quality assurance operation and the change trend, a richer and more complete quality assurance report can be generated. That is, a comparative quality assurance report can be generated based on one or more of the imaging quality assurance result and the imaging quality assurance trend result, the machine quality assurance result and the machine quality assurance trend result, and the dosimetry quality assurance result and the dosimetry quality assurance trend result.

In this example, by obtaining one or more of the historical imaging quality assurance result, the historical machine quality assurance result, and the historical dosimetry quality assurance result and comparing the one or more of the results with the imaging quality assurance result, the machine quality assurance result, and the dosimetry quality assurance result of the current quality assurance operation, respectively to determine one or more of the imaging quality assurance trend result, machine quality assurance trend result, and dosimetry quality assurance trend result. Then, according to the quality assurance result of the current quality assurance operation and the quality assurance trend result, a comparative quality assurance report is generated, which can improve the richness of the quality assurance report and provide more quality assurance information for the user.

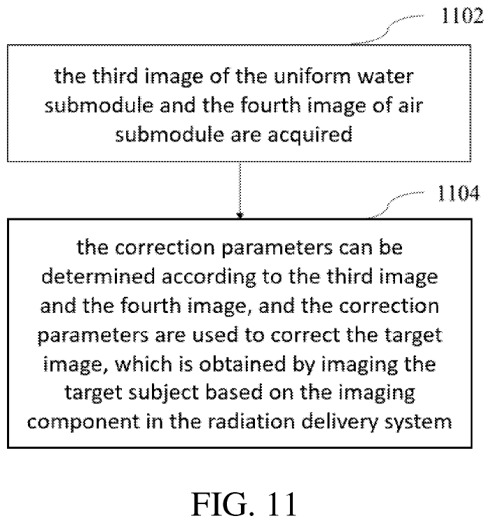

In an exemplary embodiment, the integrated quality assurance phantom as proposed above may be used not only for quality assurance operations on a radiation delivery system, but also for imaging correction. By way of example, in the case that the imaging quality assurance module of the quality assurance phantom includes a uniform water submodule (e.g., uniform water layer, uniform water block, etc.) and an air submodule. As shown in FIG. 11, the above quality assurance method may also include Steps 1102 to 1104.

At Step 1102, the third image of the uniform water submodule and the fourth image of air submodule can be acquired.

The third image can be the tomography image obtained by using the radiation delivery system to perform tomography on the uniform water sub-module of the imaging quality assurance module. The fourth image can be the tomographic image obtained by using the radiation delivery system to perform tomography on the air sub-module of the imaging quality assurance module.

For example, in the process of performing imaging performance quality assurance, sectional images of different sub-modules in the imaging quality assurance module can be obtained. The sectional images can include a third image after scanning the uniform water submodule as well as a fourth image after the air submodule is scanned.

At Step 1104, the correction parameters can be determined according to the third image and the fourth image. The correction parameters are used to correct the target image, which is obtained by imaging the target subject based on the imaging component in the radiation delivery system.

In other words, the radiation delivery system can be used to scan the target subject and obtain the target image of the target subject when it is not in quality assurance operation. Furthermore, the target image can be corrected, such as CT value correction, to obtain higher quality target image.

For example, when the CT value correction is performed, the third image of the uniform water submodule and the fourth image of air submodule can be used to determine the correction parameter. Based on the correction parameter, the CT value of the target image can be corrected, so as to obtain the corrected target image.

In this embodiment, the integrated quality assurance phantom proposed in the embodiment of this disclosure is provided, which can not only realize the quality assurance operations of multiple different performances of the radiation delivery system at the same time, but also correct the reconstructed image obtained from the scanning of the radiation delivery system, thereby further improving the quality of the reconstructed image.

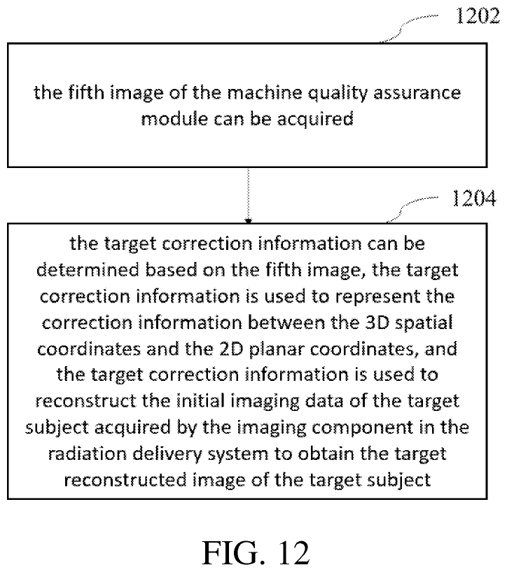

In an exemplary embodiment, the integrated quality assurance phantom as illustrated above may be used not only for quality assurance operations and imaging correction of a radiation delivery system, but also for the image reconstruction process. By way of example, in the case that the quality assurance phantom includes a machine quality assurance module, as shown in FIG. 12, above quality assurance method may also include Steps 1202 to 1204.

At Step 1202, the fifth image of the machine quality assurance module can be acquired.

The fifth image can be a 3D image obtained by imaging the machine quality assurance module using the imaging component in the radiation delivery system.

At Step 1204, the target correction information can be determined based on the fifth image. The target correction information is used to represent the correction information between the 3D spatial coordinates and the 2D planar coordinates, and the target correction information can be used to perform image reconstruction based on the initial imaging data of the target subject acquired by the imaging component in the radiation delivery system to obtain the target reconstructed image of the target subject.

In this example, the geometric correction of 3D space coordinates to 2D coordinates of flat panel detector can be realized by using metal balls in the quality assurance module of the machine. For example, by identifying the position of each metal ball in the fifth image, the 3D coordinate information of each metal ball in the 3D space coordinate system can be obtained. Then, the three-dimensional coordinate information of each metal ball can be mapped to the two-dimensional coordinate system by coordinate mapping, and the two-dimensional coordinate information of each metal ball can be obtained. Through the three-dimensional coordinate information of each metal ball and the two-dimensional coordinate information of each metal ball, the target correction information between the three-dimensional space coordinate and the two-dimensional plane coordinate can be obtained. This target correction information can be used in the image reconstruction process.

By way of example, the raw imaging data of a target subject can be obtained after the target subject is imaged by an imaging component in a radiation delivery system. Then, by image reconstruction based on the original imaging data, the target reconstructed image of the target subject can be obtained. In the process of reconstruction, the target reconstructed image of the target subject can be obtained by combining the target correction information between the three-dimensional space coordinates and the two-dimensional plane coordinates to reconstruct the original image data. Optionally, the target correction information between the 3D spatial coordinates and the 2D planar coordinates can be stored in the form of a correction table, which can be used for 3D image reconstruction.

In this example, by scanning the machine quality assurance module in the quality assurance phantom, the target correction information between the three-dimensional space coordinates and the two-dimensional plane coordinates is determined based on the fifth image obtained by scanning. The target correction information can be used in the image reconstruction process of the radiation delivery system to improve accuracy of image reconstruction.

In an exemplary embodiment, based on above integrated quality assurance phantom, a complete function of automated quality assurance and a complete quality assurance workflow are provided.

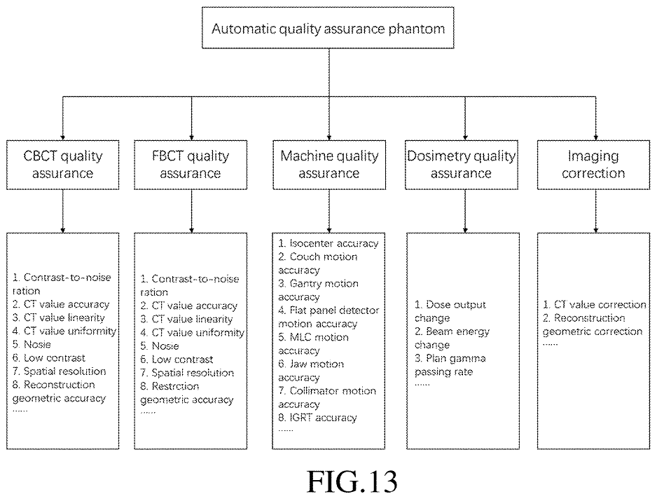

Refer to FIG. 13, which shows the quality assurance function diagram of the integrated quality assurance phantom. The quality assurance phantom can be used to evaluate the imaging quality of CBCT, FBCT and other imaging components, including contrast-to-noise ratio, CT value accuracy, CT value linearity, CT value uniformity, noise, low contrast, spatial resolution, and reconstruction geometric accuracy.

In terms of machine quality assurance, EPID in the radiation delivery system can be used to acquire images of the machine quality assurance module in the quality assurance phantom under different machine states. Thus, the machine performance metrics such as isocenter accuracy, support platform motion accuracy, gantry motion accuracy, flat panel detector motion accuracy, MLC motion accuracy, jaw motion accuracy, primary collimator motion accuracy, IGRT accuracy, flat panel detector dark field monitoring, and zero position repeatability of support platform can be tested.

In terms of dosimetry quality assurance, after the quality assurance phantom is moved out of the radiation field of the radiation delivery system, the beam output dose and energy changes could be calculated by measuring the dose output and dose distribution curves of the radiation field with different sizes. The gamma pass rate can be calculated by performing a specific quality assurance plan to determine the overall machine performance.

For image correction, the uniform water sub-module and the air sub-module in the imaging quality assurance (IQA) module could be used to correct the CT value of the reconstructed image. In addition, the metal ball in the machine quality assurance (MQA) module can be used to realize the geometric correction from 3D spatial coordinates to 2D coordinates of the flat panel detector, and the generated correction table can be used in the process of image reconstruction.

It should be noted that each performance quality assurance item in the quality assurance of the radiation delivery system may adopt a modular design. The evaluation order of each sub-module can be adjusted according to actual situation, and the evaluation nodes can be deleted or added, which can be applied to automatic quality assurance of multiple models. Furthermore, based on the quality assurance phantom described in this disclosure, combined with automatic quality assurance workflow of the device, automatic multi-type quality assurance can be realized. For example, according to the switch between quality assurance operations, the support platform can automatically operate to locate the quality assurance phantom (for example, when preparing for machine quality assurance operation or imaging quality assurance operation, the machine quality assurance module or imaging quality assurance module of the quality assurance phantom can be moved to the quality assurance position by the movement of the support platform, and when preparing for dosimetry quality assurance operation, the quality assurance phantom can be moved out of the radiation field of the radiation beam by the movement of the support platform), and then the radiation delivery system can perform the corresponding quality assurance operation, thereby realizing automatic multi-type quality assurance.

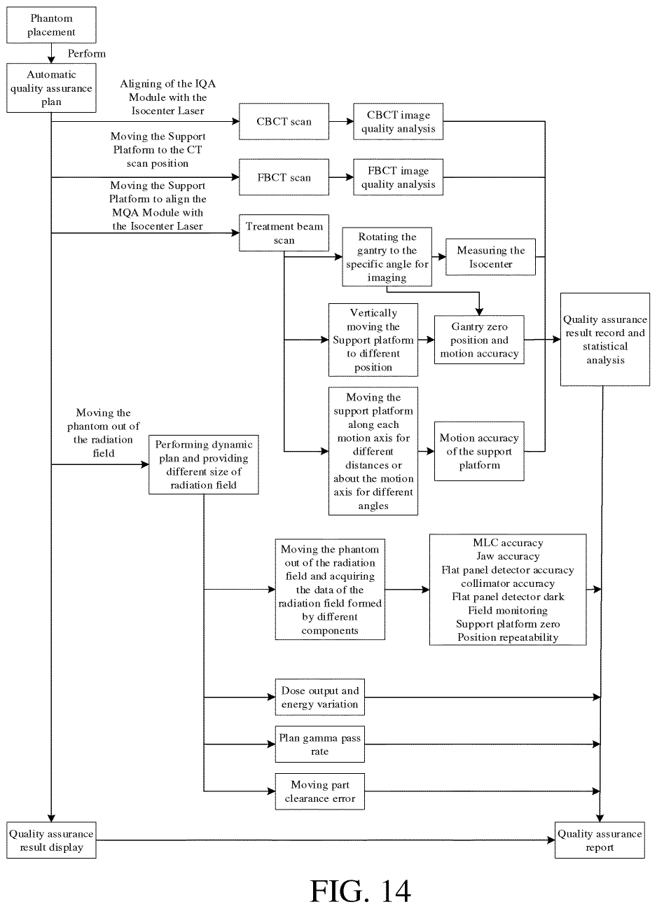

Refer to FIG. 14, an integrated and complete quality assurance workflow is shown. This workflow includes the following steps:

Firstly, the imaging quality assurance (IQA) module on the quality assurance phantom can be aligned to the laser light of the radiation delivery system. Even if the IQA module can be located at the isocenter position (above quality assurance position), automatic imaging quality assurance plan can be implemented, and the CBCT images can be acquired for imaging performance analysis. At the same time, FBCT images can be acquired (if there is no FBCT scanning device in the system, the node can be canceled in the plan), and the imaging performance can be analyzed.

Then, after the support platform moves a fixed distance automatically, the center of the machine quality assurance (MQA) module in the quality assurance phantom is aligned with the laser light. Even if the machine quality assurance (MQA) module is located at an isocenter position, the phantom images of moving parts in different motion states are obtained through the moving frame or the support platform. By calculating the change of the projection position of the metal ball in the projection diagram, the isocenter size of the machine, the zero position and motion accuracy of the gantry and the motion accuracy of the support platform can be calculated. After the image acquisition, the quality assurance phantom can be moved out of the radiation field of the radiation delivery system by automatic motion support platform. Different beam limiting components (such as MLC, jaw and/or primary collimator, etc.) can be acquired to form different radiation fields, and the motion accuracy of MLC, jaw, primary collimator and flat panel detector can be calculated, and, the flat panel detector dark field (such as dark field correction evaluation, dark field gray level, new bad point or bad line, etc.) can be monitored, and the zero repeatability of the support platform (such as couch XYZ axis) can be determined using different radiation fields.

Finally, different sizes of radiation fields can be formed through the beam limiting system, and the stability of dose output and energy change can be calculated by analyzing the dose information of different sizes of radiation fields. A specific dynamic plan can be carried out to acquire images, and the plan gamma pass rate and moving part tracking error can be calculated to evaluate the overall performance of the machine.

After the completion of the whole quality assurance process, the current and historical quality assurance result can be counted to generate the trend changes of each metric parameter, and the quality assurance report can be generated for output and archiving.

In this embodiment, a novel integrated quality assurance phantom and an automatic quality assurance planning workflow are designed. The quality assurance phantom has an innovative integrated detection module for checking mechanical and imaging properties. Based on this quality assurance phantom, the overall quality assurance task for the mechanical and imaging properties (and optional dosimetry properties) of the radiation delivery system can be achieved for the first time based on a single phantom by using the quality assurance plan workflow designed in this example.

In addition, a quality assurance data processing, analysis and result generation system which uses on the acquired images can be designed. Based on the system, accelerator quality assurance data can be automatically generated and presented in a professional report form. It solves the problem that traditional quality assurance requires strong professionalism and many repetitive operations to analyze and process the acquired data, and avoids various problems that reduce the quality of quality assurance work, such as individual experience differences, lack of some tools, and unprofessional testing methods.

Furthermore, the execution and analysis processing process of the embodiments of disclosure are fully automated. It is the first time that multi-step quality assurance tasks such as machine performance, dosimetry performance and imaging performance of a radiation delivery system are truly integrated into a single workflow for automatic implementation, and high-precision quality assurance result can be achieved based on digital images and automated analysis system. Based on automatic quality assurance, the efficiency of quality assurance work is significantly improved, the quality of quality assurance work and accuracy of data are improved, and the time-consuming and labor-consuming problems such as handling and positioning different quality assurance phantoms in different quality assurance tasks and the use of a large number of manual operations are avoided.

It should be understood that although the individual steps in the flow diagram involved in the embodiments as described above are shown sequentially as indicated by arrow, the steps are not necessarily performed sequentially in the order indicated by arrow. There are no strict order restrictions on the execution of these steps, and the steps can be performed in any other order unless explicitly stated in this article. Furthermore, at least some of the steps in the flow diagram involved in each embodiment as described above may include one or more steps or stages, which are not necessarily performed at the same time, but may be performed at different times. Moreover, the order of the steps or stages is not necessarily performed in sequence, but may be performed alternately or in turn with other steps or at least part of step or stage of other steps.

In an exemplary embodiment, a quality assurance phantom 11, 11′ is also proposed for quality assurance of a radiation delivery system, which comprises an imaging quality assurance module 111, 111′ and a machine quality assurance module 112, 112′. The imaging quality assurance module 111, 111′ can be used for imaging quality assurance operations to determine the imaging quality assurance result. The machine quality assurance module 112, 112′ can be used for the machine quality assurance operation to determine a machine quality assurance result.

In some embodiments, the quality assurance phantom 11, 11′ may further include housing 11a, 11′a, in which the imaging quality assurance module 111, 111′ and the machine quality assurance module 112, 112′ are disposed. For example, housing 11a, 11′a may surround imaging quality assurance modules 111, 111′ and machine quality assurance modules 112, 112′ to provide protection to imaging quality assurance modules 111, 111′ and machine quality assurance modules 112, 112′. The imaging quality assurance module 111, 111′ and/or the machine quality assurance module 112, 112′ can be fixed or movably arranged in the housing 11a, 11′a, so that they can be integrated into the quality assurance phantom 11, 11′ for quality assurance operation.

Referring to FIGS. 2A and 2B, in one example, the imaging quality assurance module 111 is configured to be positioned at a quality assurance location (e.g., isocenter location, etc.) in a radiation delivery system 10 when an imaging quality assurance operation is performed. In some embodiments, the quality assurance phantom 11 may be automatically moved to switch between a first state in which the imaging quality assurance module 111 is positioned at the quality assurance position and a second state in which the machine quality assurance module 112 is positioned at the quality assurance position. For example, the center point O₁ of the imaging quality assurance module 111 can be positioned at the quality assurance position. The machine quality assurance module 112 is configured to be positioned at the quality assurance position when the machine quality assurance operation is performed. For example, the center point O₂ of the machine quality assurance module 112 can be positioned at the quality assurance position.

Referring to FIG. 2C, in another example, the imaging quality assurance module 111′ and the machine quality assurance module 112′ are configured to remain fixed in position while performing imaging quality assurance operations and machine quality assurance operations. For example, the imaging quality assurance module 111′ and the machine quality assurance module 112′ can be kept at the first and second spatial positions, respectively, corresponding to the spatial positions at which the imaging quality assurance module 111′ and the machine quality assurance module 112′ are respectively positioned when the quality assurance phantom 11′ is positioned at a fixed position corresponding to the quality assurance position. As an example, when the quality assurance phantom 11′ is positioned at a fixed position corresponding to the quality assurance position, the positioning point O′ of the quality assurance phantom 11′ in FIG. 2C coincides with the quality assurance position.

By way of example, the imaging quality assurance module 111, 111′ may include one or more of a uniform water submodule, an air submodule, a contrast-to-noise ratio submodule, a low-contrast submodule, and a spatial resolution submodule. Different sub-modules in the imaging quality assurance module 111, 111′ can be used to analyze different imaging performance parameters.

By way of example, the quality assurance phantom 11, 11′ is configured to perform the steps of quality assurance method 200 as described above, that is, the quality assurance operation is performed on the radiation delivery system 10 with the quality assurance phantoms 11, 11′.

Based on the same inventive concept, embodiments of the present disclosure also provide a quality assurance device for implementing the quality assurance method described above. The implementation of the solution provided by the device is similar to the implementation described in above methods, so the specific limitations in the embodiments of one or more quality assurance devices provided below can be described in above for the quality assurance methods and will not be described here.

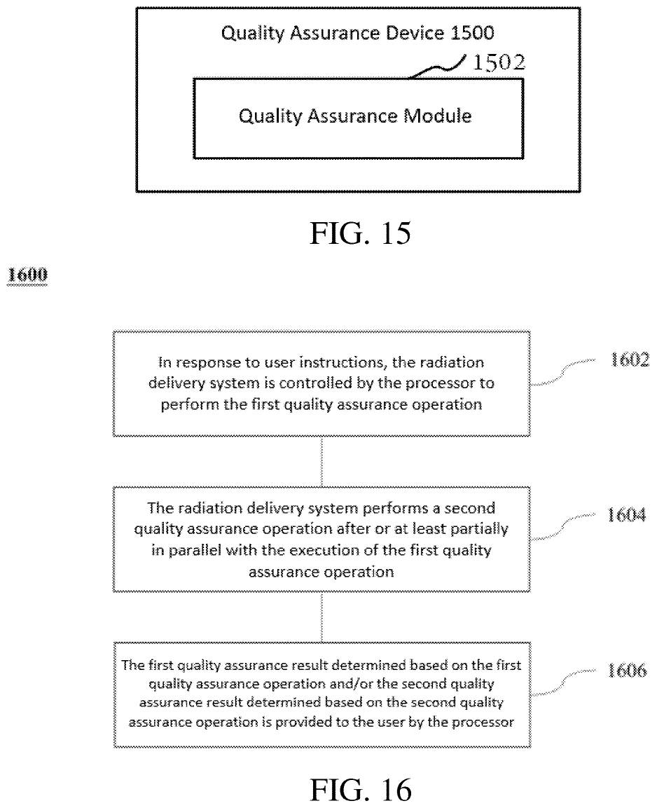

In an exemplary embodiment, as shown in FIG. 15, a quality assurance device is provided to perform quality assurance on a radiation delivery system using a quality assurance phantom comprising an imaging quality assurance module and a machine quality assurance module, the quality assurance device comprising a quality assurance module 1502 used to perform the first quality assurance operation to determine the first quality assurance result and perform the second quality assurance operation to determine the second quality assurance result, wherein the first quality assurance operation is one of the imaging quality assurance operation and the machine quality assurance operation, and the second quality assurance operation is the other one of the imaging quality assurance operation and the machine quality assurance operation. Between the first quality assurance operation and the second quality assurance operation, the quality assurance phantom is automatically moved from the first position corresponding to the first quality assurance operation to the second position corresponding to the second quality assurance operation, or remains at a fixed position.

In one embodiment, the quality assurance module 1502 is used to position the imaging quality assurance module at the quality assurance position of the radiation delivery system and the imaging quality assurance operation is performed to obtain the imaging quality assurance result, and the quality assurance phantom is moved to position the machine quality assurance module at the quality assurance position, and the machine quality assurance operation is performed to determine a machine quality assurance result; alternatively, the quality assurance module 1502 is used to position the machine quality assurance module at the quality assurance position of the radiation delivery system and the machine quality assurance operation is performed to obtain the machine quality assurance result; and the quality assurance phantom is moved to position the imaging quality assurance module at the quality assurance position, and the imaging quality assurance operations is performed to determine the imaging quality assurance result.

In another embodiment, the quality assurance module 1502 is used to position the quality assurance phantom at the quality assurance position of the radiation delivery system, and the imaging quality assurance operation is performed to determine the imaging quality assurance result, and the quality assurance phantom is kept in the quality assurance position, and the machine quality assurance operation is performed to determine a machine quality assurance result; or the quality assurance module 1502 is used to position the quality assurance phantom at the quality assurance position of the radiation delivery system, and the machine quality assurance operation is performed to determine the machine quality assurance result; and the quality assurance phantom is kept in the quality assurance position, and the imaging quality assurance operation is performed to determine the imaging quality assurance result.

In one embodiment, in the case that the quality assurance method is an automatic quality assurance method implemented based on the processor, the quality assurance module 1502 is used to control the movement of the support platform to position the imaging quality assurance module on the support platform at the quality assurance position of the radiation delivery system and perform the imaging quality assurance operation to determine the imaging quality assurance result; and control the movement of the support platform to position the machine quality assurance module on the support platform at the quality assurance position and perform the machine quality assurance operation to determine a machine quality assurance result.

In one embodiment, the imaging quality assurance module includes at least one submodule, each of which is used to be imaged to determine different imaging performance parameters. The quality assurance module 1502 includes:
a first acquisition unit used to acquire the first image of each sub-module; and
a first analysis unit used to analyze the imaging performance of the first image of each sub-module, and determine the imaging quality assurance result including one or more imaging performance parameters.

In one embodiment, the quality assurance module 1502, further comprises:
a second acquisition unit used to adjust the motion state of one or more moving parts in the radiation delivery system according to the predetermined machine parameters, and acquire the second image of the quality assurance module of the machine under different motion states; and
a second analysis unit used to analyze each second image according to the corresponding analysis strategy of the second image, and determine a machine quality assurance result including one or more machine performance parameters.

In one embodiment, a machine quality assurance result include the motion accuracy of the beam limiting component in the radiation delivery system. The quality assurance module 1502 is also used to control the beam limiting component to form different radiation fields and determine the motion accuracy of the beam limiting component according to the measured radiation fields when the quality assurance phantom is positioned outside the radiation field of the radiation delivery system.

In one embodiment, the quality assurance module 1502 is also used to position the quality assurance phantom at the radiation field of the radiation delivery system and perform a dosimetry quality assurance operation to determine the dosimetry quality assurance result.

In one embodiment, the quality assurance module 1502, further comprises:
a first acquisition unit used to control the beam limiting component in the radiation delivery system to form different radiation fields and obtain the dose information corresponding to different radiation fields; and
a third analysis unit used to analyze each dose information and determine a dosimetry quality assurance result including one or more dosimetry performance parameters.

In one embodiment, the quality assurance device also includes a report generation module for generating quality assurance reports based on one or more of the imaging quality assurance result, a machine quality assurance result, and a dosimetry quality assurance result. The report generation module includes:
a second acquisition unit used to obtain one or more of the historical imaging quality assurance result, historical machine quality assurance result, and historical dosimetry quality assurance result; and
one or more of the first contrast unit, the second contrast unit, and the third contrast unit, wherein the first comparison unit is used to perform the comparison analysis on the historical imaging quality assurance result and imaging quality assurance result, and determine the trend of imaging quality assurance result; the second comparison unit is used to perform the comparison analysis on the historical machine quality assurance result and a machine quality assurance result, and determine the trend of a machine quality assurance result, and the third comparison unit is used to perform the comparison analysis on the historical dosimetry quality assurance result and a dosimetry quality assurance result and determine the trend result of dosimetry quality assurance; and
a report generation unit is used to generate a quality assurance report based on one or more of the imaging quality assurance result, machine quality assurance result, and dosimetry quality assurance result, as well as the corresponding one or more of the imaging quality assurance trend result, machine quality assurance trend result, and dosimetry quality assurance trend result.

In one embodiment, the imaging quality assurance module includes a uniform water submodule and an air submodule; the quality assurance device also includes:
a first acquisition module used to acquire the third image of the uniform water sub-module and the fourth image of air sub-module; and
a determination module used to determine the correction parameters according to the third image and the fourth image; the correction parameters are used to correct the target image, which is obtained by imaging the target subject based on the imaging component in the radiation delivery system.

In one embodiment, the quality assurance device also includes:
a second acquisition module used to acquire the fifth image of the machine quality assurance module; and
a determination module used to determine the target correction information based on the fifth image; the target correction information is used to represent the correction information between the 3D spatial coordinates and the 2D planar coordinates, and the target correction information is used to reconstruct the original imaging data of the target subject acquired by the imaging component in the radiation delivery system to obtain the target reconstructed image of the target subject.

Each module of above quality assurance device can be implemented in whole or in part by software, hardware and their combination. The above modules can be embedded in or independent of the processor in the computer device in the form of hardware, or can be stored in the memory of the computer device in the form of software, so as to facilitate the processor to call and execute the corresponding operations of above modules.

In an exemplary embodiment, a quality assurance method 1600 is provided. The quality assurance method 1600 can be applied in the application environment as shown in FIG. 1B. The quality assurance method 1600 may be executed by a processor within and/or outside the radiation delivery system 10.

At step 1602, in response to user instructions, the radiation delivery system 10 can be controlled by the processor to perform the first quality assurance operation. The user may enter user instructions via a user control terminal (e.g., touch screen, keyboard, microphone, mouse, etc.) of the radiation delivery system 10. As an example, a user can enter a user instruction by clicking a virtual button with the mouse.

At step 1604, the radiation delivery system 10 performs a second quality assurance operation after or at least partially in parallel with the execution of the first quality assurance operation. In some embodiments, the second quality assurance operation begins after the first quality assurance operation has been completed. In other embodiments, while the first quality assurance operation is performed, the radiation delivery system 10 begins to perform a second quality assurance operation to shorten the overall quality assurance process time. For example, while the machine quality assurance operation is performed, the imaging component that has completed the machine quality assurance operation or does not need to perform the machine quality assurance operation can perform the imaging quality assurance operation on the imaging quality assurance module 111 of the quality assurance phantom 11.

Here, the first quality assurance operation can be one of the imaging quality assurance operation and machine quality assurance operation, and the second quality assurance operation can be the other one of the imaging quality assurance operation and machine quality assurance operation. Between the first quality assurance operation and the second quality assurance operation, the quality assurance phantom 11 could be automatically moved from the first position corresponding to the first quality assurance operation to the second position corresponding to the second quality assurance operation, or remain in a fixed position. For example, the quality assurance phantom 11 for the imaging quality assurance operation and the machine quality assurance operation may be automatically moved or remain in a fixed position during the period from the completion of the first quality assurance operation to the beginning of the second quality assurance operation.

Optionally, at step 1606, the first quality assurance result determined based on the first quality assurance operation and/or the second quality assurance result determined based on the second quality assurance operation may be provided to the user by the processor. In some embodiments, the first quality assurance result and/or the second quality assurance result may be displayed to the user through a display. In other embodiments, the first quality assurance result and/or the second quality assurance result may be notified to the user via loudspeakers or alarms. In still other embodiments, the first quality assurance result and/or the second quality assurance result can be communicated to the user by printing out a paper report by a printer or sending an electronic report to the user terminal.

Optionally, a dosimetry quality assurance operation may also be performed before, between, or after the execution of the first quality assurance operation. For example, the quality assurance phantom can be positioned outside the radiation field of the radiation delivery system, and the dosimetry quality assurance operation can be performed to determine the dosimetry quality assurance result.

In some embodiments, in quality assurance method 1600, at least the process from the first to the second quality assurance operation is performed automatically.

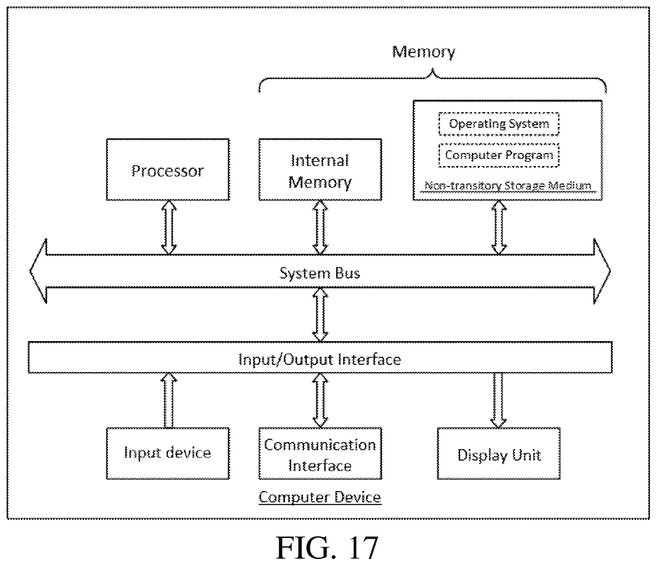

In an exemplary embodiment, a computer device is provided, which may be a radiation delivery device in a radiation delivery system, or a quality assurance terminal which is communicated and connected to the radiation delivery system. Taking the quality assurance terminal as an example, internal structure diagram of the computer device may be shown in FIG. 17. The computer device can include a processor, a memory, an input/output interface, a communication interface, a display unit and an input device. The processor, memory and input/output interface are connected through the system bus, and the communication interface, display unit and input device are connected to the system bus through the input/output interface. The processor of the computer device is used to provide computing and control capabilities. The memory of the computer device includes a non-volatile storage medium and an internal memory. This non-volatile storage medium stores the operating system and computer programs. This internal memory provides an environment for operating systems and computer programs to run in a non-transitory storage medium. The input/output interface of the computer device is used to exchange information between the processor and the external device. The communication interface of the computer device is used to communicate with external terminals either wired or wirelessly through WIFI, mobile cellular networks, NFC (near field communication), or other technologies. The computer program is executed by the processor to implement a quality assurance method. The display unit of the computer device is used to form a visually visible pattern, which may be a display screen, a projection device, or a virtual reality imaging device. The display screen can be an LCD screen or an e-ink screen. The input device of the computer device can be a touch layer covered on the display screen, a button, trackball or trackpad set on the shell of the computer device, and an external keyboard, trackpad or mouse.

The skilled person in art can understand that the structure shown in FIG. 16 is only a block diagram of the part of the structure related to the scheme of disclosure and does not constitute a limitation of the computer device on which the scheme of disclosure is applied. The specific computer device may include more or fewer parts than shown in the figure, or combine some parts, or have different parts arrangement.

In an exemplary embodiment, a computer device is provided, including a processor and a memory in which a computer program is stored, and when executed by the processor, the computer program implements the steps of the quality assurance method in any of above embodiments.

In one embodiment, a computer readable storage medium is provided on which a computer program is stored, and when executed by the processor, the computer program implements the steps of implementing the quality assurance method in any of above embodiments.

In one embodiment, a computer program product is provided, the computer program product including a computer program which, when executed by a processor, implements the steps of the quality assurance method in any of above embodiments.

It should be noted that the data involved in this disclosure (including but not limited to the data used for analysis, the data stored, the data displayed, etc.) are all information and data fully authorized by the parties, and the acquirion, use and processing of the relevant data shall comply with relevant regulations.

One of ordinary skill in art will understand that the process of implementing all or part of the methods of above embodiments can be accomplished by instructing the relevant hardware through a computer program, which may be stored in a non-transitory computer readable storage medium, and which, when executed, may include the process as examples of the methods above. Among others, any reference to a memory, database or other medium used in each of the embodiments provided in this disclosure may include at least one of non-transitory and transitory memory. A non-volatile memory may include a Read-Only Memory, ROM), magnetic tape, floppy disk, flash Memory, optical memory, high-density embedded non-volatile memory, resistive memory (ReRAM), Magnetoresistive Random Access memory (Magnetoresistive Random Access Memory, MRAM), Ferroelectric Random Access Memory (FRAM), Phase Change Memory (PCM), graphene memory, etc. Volatile Memory may include Random Access Memory (RAM) or external cache memory. As an illustration, but not a limitation, RAM can be in a variety of forms, such as Static Random Access Memory (SRAM) or Dynamic Random Access Memory (DRAM). The databases involved in each embodiment provided in disclosure may include at least one of relational and non-relational databases. As non-limiting example, non-relational databases may include distributed databases based on blockchain, etc. As non-limiting example, the processors involved in each of the embodiments provided in this disclosure may be general-purpose processors, central processing units, graphics processors, digital signal processors, programmable logics, quantum computing-based data processing logics, etc.

The technical features of above embodiments may be arbitrarily combined. For the sake of bretness of the description, all possible combinations of the technical features of above embodiments are not described, however, as long as there is no contradiction in the combination of these technical features, they should be considered as being within the scope of the description.

The above embodiments only express several embodiments of the present disclosure, and the description is more specific and detailed, but it is not therefore understood to be a limitation of the scope of the present disclosure. It should be noted that, for ordinary technicians in this field, certain deformations and improvements can be made without leaving the idea of this disclosure, which are within the scope of protection of this disclosure. Therefore, the scope of protection of this disclosure shall be subject to attached claims.

Further, it will be appreciated by one skilled in art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

Furthermore, the recited order of processing elements or sequences, or the use of numbers, letters, or other designations therefore, is not intended to limit the claimed processes and methods to any order except as may be specified in the claims. Although above disclosure discusses through various examples what is currently considered to be a variety of useful embodiments of the disclosure, it is to be understood that such detail is solely for that purpose, and that appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of disclosure disclosed herein are illustrative of the principles of the embodiments of disclosure. Other modifications that may be employed may be within the scope of disclosure. Thus, by way of example, but not of limitation, alternative configurations of the embodiments of disclosure may be utilized in accordance with the teachings herein. Accordingly, embodiments of the present disclosure are not limited to that precisely as shown and described.

1. A quality assurance method for performing quality assurance on a radiation delivery system, the method comprising:
performing a first quality assurance operation using a quality assurance phantom; and
performing a second quality assurance operation using the quality assurance phantom,
wherein the first quality assurance operation is one of an imaging quality assurance operation and a machine quality assurance operation, and the second quality assurance operation is the other one of the imaging quality assurance operation and the machine quality assurance operation, and
between the first quality assurance operation and the second quality assurance operation, the quality assurance phantom is automatically moved from a first position corresponding to the first operation to a second position corresponding to the second operation, or remains at a fixed position.

2. The quality assurance method of claim 1, wherein the quality assurance phantom includes an imaging quality assurance module for the imaging quality assurance operation and a machine quality assurance module for the machine quality assurance operation.

3. The quality assurance method of claim 2, wherein the quality assurance method includes:
positioning the imaging quality assurance module at a quality assurance position of the radiation delivery system and performing the imaging quality assurance operation to determine an imaging quality assurance result; and automatically moving the quality assurance phantom to position the machine quality assurance module at the quality assurance position and performing the machine quality assurance operation to determine a machine quality assurance result, or
positioning the machine quality assurance module at the quality assurance position of the radiation delivery system and performing the machine quality assurance operation to determine a machine quality assurance result; and automatically moving the quality assurance phantom to position the imaging quality assurance module at the quality assurance position and performing the imaging quality assurance operation to determine an imaging quality assurance result.

4. The quality assurance method of claim 3, wherein the imaging quality assurance module includes at least one sub-module, each sub-module being configured for being imaged to determine different imaging performance parameters, and the performing the imaging quality assurance operation to determine an imaging quality assurance result includes:
acquiring a first image of each sub-module; and
performing imaging performance analysis on the first images to determine the imaging quality assurance result including one or more imaging performance parameters.

5. The quality assurance method of claim 3, wherein performing the machine quality assurance operation to determine a machine quality assurance result includes:
adjusting the motion state of one or more moving parts in the radiation delivery system according to predetermined machine parameters;
acquiring second images of the machine quality assurance module under different motion states; and
analyzing the second images to determine the machine quality assurance result including one or more machine performance parameters.

6. The quality assurance method of claim 2, wherein the imaging quality assurance module includes a uniform water sub-module and an air sub-module; the quality assurance method further comprising:
acquiring a third image of the uniform water sub-module and a fourth image of the air sub-module; and
determining a correction parameter based on the third image and the fourth image, the correction parameters being used for correcting a target image, the target image being obtained by imaging a target subject using the imaging component in the radiation delivery system.

7. The quality assurance method of claim 2, further comprising:
acquire a fifth image of the machine quality assurance module;
determining target correction information based on the fifth image; the target correction information being used for characterizing the correction information between three-dimensional spatial coordinates and two-dimensional planar coordinates, and the target correction information being used for perform image reconstruction on the initial imaging data of the target subject acquired by the imaging component in the radiation delivery system to obtain the target reconstructed image of the target subject.

8. The quality assurance method of claim 2, wherein the quality assurance method comprising:
positioning the quality assurance phantom at a quality assurance position of the radiation delivery system and performing the imaging quality assurance operation to determine the imaging quality assurance result; and maintaining the phantom at the quality assurance position and performing the machine quality assurance operation to determine the machine quality assurance result, or
positioning the quality assurance phantom at the quality assurance position and performing the machine quality assurance operation to determine a machine quality assurance result; and
maintaining the phantom at the quality assurance position and performing the imaging quality assurance operation to determine an imaging quality assurance result.

9. The quality assurance method of claim 2, wherein the quality assurance method includes:
in response to a user instruction input by a user, controlling the radiation delivery system to perform the first quality assurance operation;
after completion of the first quality assurance operation, controlling the support platform to move the quality assurance phantom disposed thereon to the second position, or maintaining the phantom at the first position, and automatically controlling the system to perform the second quality assurance operation; and
after completion of the second operation, providing feedback information to the user via a user interface.

10. The quality assurance method of claim 1, wherein further comprising:
positioning the quality assurance phantom outside the radiation field of the radiation delivery system and performing a dosimetry quality assurance operation to determine a dosimetry quality assurance result.

11. The quality assurance method of claim 1, wherein the quality assurance method further comprising:
generating a quality assurance report based on one or more of an imaging quality assurance result, a machine quality assurance result, and a dosimetry quality assurance result, which includes:
obtaining one or more of a historical imaging quality assurance result, a historical machine quality assurance result, and a historical dosimetry quality assurance result;
performing one or more of following steps (i) to (iii): (i) comparing the historical imaging quality assurance result and the imaging quality assurance result to determine an imaging quality assurance trend result, (ii) comparing the historical machine quality assurance result and the machine quality assurance result to determine a machine quality assurance trend result, and (iii) comparing the historical dosimetry quality assurance result and the dosimetry quality assurance to determine a dosimetry quality assurance trend result; and
generating a quality assurance report based on one or more of the imaging quality assurance result, the machine quality assurance result and the dosimetry quality assurance result, and one or more of the imaging quality assurance trend result, the machine quality assurance trend result and the dosimetry quality assurance result.

12. The quality assurance method of claim 1, wherein at least the procedure from the first quality assurance operation to the second quality assurance operation is performed automatically.

13. The quality assurance method of claim 2, wherein the imaging quality assurance module includes one or more of: a uniform water sub-module, an air sub-module, a contrast-to-noise ratio sub-module, a low-contrast sub-module, and a spatial resolution sub-module.

14. The quality assurance method of claim 1, wherein the quality assurance phantom is automatically moved to switch between a first state where the imaging module is positioned at the quality assurance position and a second state where the machine module is positioned at the quality assurance position.

15. A quality assurance method using a quality assurance phantom for a radiation delivery system, the quality assurance method comprising:
in response to a user instruction, controlling, by a processor, the radiation delivery device to perform a first quality assurance operation; and
after or at least partially concurrently with the first operation, performing a second quality assurance operation,
wherein the first operation is one of an imaging quality assurance operation and a machine quality assurance operation, and the second operation is the other one of the imaging quality assurance operation and the machine quality assurance operation, and
between the first operation and the second operation, the quality assurance phantom is automatically moved from a first position corresponding to the first operation to a second position corresponding to the second operation, or remains at a fixed position.

16. The quality assurance method of claim 15, wherein the quality assurance phantom includes an imaging quality assurance module for the imaging quality assurance operation and a machine quality assurance module for the machine quality assurance operation.

17. The quality assurance method of claim 16, wherein the quality assurance method includes:
positioning the imaging quality assurance module at a quality assurance position of the radiation delivery system and performing the imaging quality assurance operation to determine an imaging quality assurance result; and automatically moving the quality assurance phantom to position the machine quality assurance module at the quality assurance position and performing the machine quality assurance operation to determine a machine quality assurance result, or
positioning the machine quality assurance module at the quality assurance position of the radiation delivery system and performing the machine quality assurance operation to determine a machine quality assurance result; and automatically moving the quality assurance phantom to position the imaging quality assurance module at the quality assurance position and performing the imaging quality assurance operation to determine an imaging quality assurance result.

18. The quality assurance method of claim 16, wherein the imaging quality assurance module includes at least one sub-module, each sub-module being configured for being imaged to determine different imaging performance parameters, and the performing the imaging quality assurance operation to determine an imaging quality assurance result includes:
acquiring a first image of each sub-module; and
performing imaging performance analysis on the first images to determine the imaging quality assurance result including one or more imaging performance parameters.

19. The quality assurance method of claim 16, wherein the quality assurance method comprising:
positioning the quality assurance phantom at a quality assurance position of the radiation delivery system and performing the imaging quality assurance operation to determine the imaging quality assurance result; and maintaining the phantom at the quality assurance position and performing the machine quality assurance operation to determine the machine quality assurance result, or
positioning the quality assurance phantom at the quality assurance position and performing the machine quality assurance operation to determine a machine quality assurance result; and
maintaining the phantom at the quality assurance position and performing the imaging quality assurance operation to determine an imaging quality assurance result.

20. A non-transitory computer-readable storage medium having a computer program stored thereon, characterized in that when the computer program is executed by a processor, the steps of the quality assurance method of claim 1.
